(19) United States
(12) Patent Application Publication
(10) Pub. No.: US 2019/0016810 A1
(43) Pub. Date: Jan. 17, 2019

(54) METHODS FOR TREATING OR PREVENTING GRAFT-VERSUS-HOST DISEASE INVOLVING THE ADMINISTRATION OF ANTI-CCR5 RECEPTOR AGENTS

(71) Applicant: CytoDyn Inc., Vancouver, WA (US)

(72) Inventor: Denis R. Burger, Vancouver, WA (US)

(21) Appl. No.: 15/977,929

(22) Filed: May 11, 2018

Related U.S. Application Data
(60) Provisional application No. 62/585,974, filed on Nov. 14, 2017.
(60) Provisional application No. 62/504,749, filed on May 11, 2017.

(51) Int. Cl. C07K 16/28; A61P 37/00

(52) U.S. Cl. CPC C07K 16/2866; A61K 2039/505; C07K 2317/76; A61P 37/00

(57) ABSTRACT

The present invention provides for inhibition or blockade of immunomodulatory cell receptors to treat or prevent graft-versus-host disease (GVHD). Thus, the invention relates generally to compositions and methods of using anti-CCR5 cell receptor binding agents, such as PRO 140, to treat or prevent GVHD.

### BACKGROUND

### Technical Field

The present disclosure relates to the use of competitive inhibitors of the CCR5 receptor, such as the monoclonal antibody PRO 140, in the treatment or prevention of graft-versus-host disease (GVHD).

### Description of the Related Art

GVHD is a major complication of allogeneic hematopoietic stem cell transplant (AHSCT) associated with significant morbidity and mortality. New discoveries in immunology and inflammation have expanded our understanding of GVHD, in which tissue damage from chemotherapy or radiation results in cytokine release, which activates T cells, resulting in proliferation and differentiation, trafficking to target organs, and tissue destruction and inflammation. Insights into the mechanisms of this disease relate directly to the development of preventive strategies and therapies, such as immunosuppression, T cell depletion, calcineurin inhibitors, CCR5 cell receptor antagonists, gut decontamination, extracorporeal photopheresis, and more. GVHD is typically characterized as acute GVHD or chronic GVHD and affects, for example, the gut, liver, and skin.

Inflammation may occur in response to trauma, chemical or physical injury, autoimmune responses, infectious agents, cancer, etc. Inflammation is an important component of innate immunity and is necessary for priming adaptive immunity and for the effector phase of the immune response. Soluble mediators, such as chemokines, are shown to play an important role in driving the various components of inflammation, especially leukocyte influx. Chemokines and their receptors are important in many human diseases, including HIV, autoimmune disease, inflammatory disease and organ graft rejection, and can include, for example, the chemokine CCL5 (RANTES) and the cell receptor CCR5. Currently, small molecule antagonists and humanized monoclonal antibodies targeting chemokine receptors are developed by industry, and tested in animals, preclinical models, and in phase I trials. Data demonstrates that CCR5⁺ T cells are important in mediating GVHD in humans.

Chemokine receptor CCR5 has been shown to mediate murine GVHD pathogenesis. It is reported that infiltrating lymphocytes in the skin of human acute GVHD samples are predominantly CCR5⁺ T cells. Lisa Palmer, George Sale, John Balogun, Dan Li, Dan Jones, Jeffrey Molldrem, Rainer Storb, Qing Ma, Chemokine Receptor CCR5 Mediates Allo-Immune Responses in Graft-vs-Host Disease, Biol Blood Marrow Transplant. 2010 March; 16(3): 311-319, doi: 10.1016/j.bbmt2009.12.002. It has also been found that the CCR5⁺ population exhibits the characteristics of the activated effector T cell phenotype. CCR5 expression is upregulated upon allogenic stimulation, and CCR5⁺ cells are proliferating with co-expression of T cell activation markers. Furthermore, the activated T cells producing inflammatory cytokine TNFα, IL-2 or IFN-γ, are positive for CCR5. Thus, it is understood that CCR5 is a marker for GVHD effector cells, and CCR5⁺ T cells are active participants in the pathogenesis of human acute GVHD.

As noted above, immunosuppression has been used as a GVHD preventive therapy and has been the primary pharmacologic strategy to prevent GVHD. Methotrexate has been used since the 1950s as a way of shutting down T cells through inhibition of dihydrofolate reductase and production of thymidylate and purines. Anthony D. Sung and Nelson J. Chao, Concise Review: Acute Graft-Versus-Host Disease: Immunobiology, Prevention, and Treatment, Stem Cells Translations Medicine 2013; 2:25-32 (“Sung 2013”). Post-transplant cyclophosphamide is another method of eliminating rapidly dividing T cells that shows promise in recent clinical trials. The calcineurin inhibitors cyclosporine and tacrolimus inhibit T cell proliferation; combinations with methotrexate have successfully been used since the 1970s and are the cornerstone of most prophylactic regimens. However, these agents have numerous side effects, including delayed cell count and immunological recovery, thrombotic microangiopathy, and posterior reversible encephalopathy syndrome. The inosine monophosphate dehydrogenase inhibitor mycophenolate mofetil and the mammalian target of rapamycin (mTOR) inhibitor sirolimus have been proposed as alternate agents, but there is no clear consensus on optimal drug combination, dosing, or timing.

Other drugs attempt to target cytokine/chemokine-receptor interactions that appear integral to development of GVHD. Some success has been reported with maraviroc, a small molecule CCR5 antagonist that reportedly blocks T cell chemotaxis and dramatically decreases the incidence of gastrointestinal and liver GVHD. At the same time, despite the central role of cytokines IL-1 and TNF-α, drugs that block these pathways (etanercept, infliximab) have failed to improve rates of acute GVHD. Alternative methods of reducing GVHD involve targeting and dampening the cytokine storm that triggers the cascade of downstream events leading to GVHD. For example, reduced intensity conditioning causes less tissue damage and has been shown to reduce GVHD. Additionally, gut decontamination with ciprofloxacin and metronidazole has been shown to decrease the risk of acute GVHD compared with ciprofloxacin alone. However, attempts to preserve epithelial integrity with keratinocyte growth factor (palifermin) did not decrease the risk of GVHD, although palifermin has been shown to reduce the risk of mucositis.

Given the central role of T cells in GVHD, T cell depletion (TCD) has been studied since the 1980s as a preventative strategy. This can be done with physical techniques, such as ex vivo counterflow centrifugal elutriation or soybean lectin agglutination and E-rosetting, or by immunological methods, such as ex vivo or in vivo administration of anti-sera (anti-thymocyte globulin) or monoclonal antibodies; positive selection techniques can also isolate CD34+ cells ex vivo, allowing T cells to be discarded. Randomized trials have shown that although TCD successfully decreases the risk of GVHD, the risks of graft failure, disease relapse, and opportunistic infections are increased. However, some of these risks can be mitigated (e.g., higher CD34+ cell dose to promote engraftment, antibiotic prophylaxis to prevent opportunistic infections), and more recent single-arm trials have shown 3-year disease-free survival approaching 60%. Furthermore, T cell depletion strategies such as in vivo administration of the anti-CD52 antibody alemtuzumab can facilitate transplants from HLA-mismatched haploidentical donors without significant GVHD, opening up transplant options to patients without HLA-matched donors.

CCR5 is considered one of the “inflammatory” chemokine receptors regulated by proinflammatory stimuli to orchestrate immune responses, in comparison to the “homeostatic” group which are important in immune surveillance such as CCR7. Inflammatory chemokine receptors are upregulated during tissue damage or inflammation, and are involved in T cell polarization. It has been found that activated T cells producing cytokine TNFα, IL-2 or IFN-γ are positive for CCR5. Gene expression profiles have shown that CCR5 was upregulated during aGVHD in humans. Data further demonstrated that CCR5 expression is on activated and upregulated and proliferating T cells upon allogeneic stimulation. Indeed, it has been reported that the expression of CCR5 on T cells is restricted to the proliferating T lymphocytes. In contrast, CCR7 was found within both proliferating and non-proliferating T cells, and there was no significant increase in the expression of CCR7. Interestingly, CXCR3, another inflammatory chemokine receptor, does not show the same restriction to proliferating cells as CCR5. Although almost all proliferating T cells were positive for CXCR3, there were high percentages of non-proliferating T cells expressing this chemokine receptor. Thus, available information suggests a critical role of CCR5 expression in alloimmune responses and GVHD. Indeed, in skin and lip biopsies from aGVHD patients examined in situ for CCR5 expression it was found that the majority of infiltrating T cells were positive for CCR5.

CCR5 is highly upregulated on both CD4⁺(Th1) T cells and activated antigen specific CD8⁺ T cells. Using an intracellular cytokine assay, it has been found that activated CD4⁺ T cells producing inflammatory cytokine TNFα, IL-2 or IFN-γ, were positive for CCR5. In addition, the TNFα and IL-2 producing CD8⁺ T cells also expressed CCR5. This observation supports the notion that CCR5 is a marker for effector T cells that actively participate in the “cytokine storm” of GVHD.

Further, a protective effect of the CCR5 deletion mutation in GVHD has been demonstrated. A small cohort study found that, in the absence of CCR5 on donor cells, there is a decreased incidence of GVHD and increased relapse rate in recipients receiving HLA-matched unrelated marrow donors. In particular, a significant reduction of skin GVHD was shown.

It is known that chemokines and their receptors play an important role in regulating leukocyte migration and activation. Chemokines bind to their receptors, which are expressed on many cell types, including, for example, leukocytes, endothelial cells, fibroblasts, epithelial, smooth muscle, and parenchymal cells. Chemokines play an important role in leukocyte biology, by controlling cell recruitment and activation in basal and in inflammatory circumstances. In addition, because chemokine receptors are expressed on other cell types, chemokines have multiple other roles, including angiogenesis, tissue and vascular remodeling, pathogen elimination, antigen presentation, leukocyte activation and survival, chronic inflammation, tissue repair/healing, fibrosis, embryogenesis, tumorigenesis, etc.

CCL5 (C-C chemokine ligand 5), an inflammatory chemokine also known as regulated upon activation and normal T cell expressed and secreted (RANTES), plays an important role in the above-noted immunologic mechanisms. CCL5 acts as a key regulator of T cell migration to inflammatory sites, directing migration of T cells to damaged or infected sites. CCL5 also regulates T cell differentiation. Many biologic effects of chemokines are mediated by their interaction with chemokine receptors on cell surfaces. In the present invention, the most relevant known receptor for CCL5 is the CCR5 receptor; however, CCR1 and CCR3 are also known CCL5 receptors and CCR4 and CD44 are auxiliary receptors. Tamamis et al., Elucidating a KeyAnti-HIV-1 and Cancer-Associated Axis: The Structure of CCL5 (Rantes) in Complex with CCR5, SCIENTIFIC REPORTS, 4: 5447 (2014).

The formation of the CCL5 ligand and CCR5 receptor complex causes a conformational change in the receptor that activates the subunits of the G-protein, inducing signaling and leading to changed levels of cyclic AMP (cAMP), inositol triphosphate, intracellular calcium, and tyrosine kinase activation. These signaling events cause cell polarization and translocation of the transcription factor NF-kB, which results in the increase of phagocytic ability, cell survival, and transcription of proinflammatory genes. Once G-protein dependent signaling occurs, the CCL5/CCR5 receptor complex is internalized via endocytosis.

The inventor previously showed that the monoclonal antibody PRO 140 does not affect cAMP levels when added to CD4⁺ T cells alone, but does diminish the effect of CCL5 on cAMP levels when administered with CCL5. WO2016/210130. Similarly, the inventor also previously showed that, although PRO 140 alone does not affect chemotaxis of CHO-K1 cells, PRO 140 does reduce CCL5-induced chemotaxis when administered with CCL5. WO2016/210130. These studies indicate that PRO 140 does not have agonist activity for CCR5 but does act as a competitive inhibitor with CCL5 for binding to CCR5.

The inventor has also found that PRO 140 alone does not affect tyrosine kinase phosphorylation downstream of CCR5 receptor signaling in T cells in vitro, and that it also does not inhibit phosphorylation of such kinases by CCL5. These results provide evidence that the role of PRO 140 in modulating the CCR5/CCL5 axis relative to RANTES is inconsistent, i.e., that it blocks or inhibits only some of the downstream activities that would otherwise result from CCL5/CCR5 binding. Thus, it is shown that PRO 140's activity as a CCL5 competitive binding inhibitor has a mixed impact on CCL5's ability to participate in downstream activity conventionally associated with the CCR5/CCL5 axis. Further elucidation of the role of PRO 140 and its immunomodulatory effects is the subject of continued investigation.

It has been found that the most potently antiviral anti-CCR5 monoclonal antibodies including, for example, PRO 140, bind CCR5 receptor amino acid residues in EL2 alone or in combination with Nt residues. It has also been determined that the CCR5 receptor binding sites for anti-CCR5 monoclonal antibodies are distinct from those of small-molecule CCR5 antagonists. That is, available small-molecule CCR5 antagonists, such as maraviroc, bind the hydrophobic cavity formed by the transmembrane helices, i.e., not the extracellular Nt or loop regions. The amino acid residue E283 in the seventh transmembrane region has been specifically identified as a principle site or interaction for small molecules, and maraviroc and vicriviroc have been found to bind to identical sets of CCR5 receptor amino acids. Olson et al., CCR5 Monoclonal Antibodies for HIV-1 Therapy, CURR. OPIN. HIV AIDS, March, 4(2): 104-111 (2009). It has also been reported, however, that the CCL5 ligand and maraviroc dock on the CCR5 receptor by sharing two receptor sites: the Nt and the ECL2, and that synthetic CCL5-derived peptides may also be used to block the CCR5 receptor. Secchi et al., Combination of the CCL5 -Derived Peptide R4.0 with Different HIV-1 Blockers Reveals Wide Target Compatibility and Synergic Cobinding to CCR5, ANTIMICROB AGENTS CHEMOTHER., 58(10): 6215-6223 (2014).

In vitro studies have been conducted to investigate the effects of CCR5 receptor blockade by maraviroc on activated human T cells on potential immunological mechanisms. It was found that blocking CCR5 by maraviroc not only can block CCR5 and CCR2 internalization processes induced by CCL5 and CCL2, but can also inhibit T cell chemotactic activities toward their cognate ligands, respectively. Further, blocking CCR5 with maraviroc at high doses tends to decrease production of TNF-α and IFN-γ. It was also noted that the effect of maraviroc on CCR5 was temporary and reversible. Yuan et al., In Vitro Immunological Effects of Blocking CCR5 on T Cells, INFLAMMATION, 38(2): 902-910 (2015); see Arberas et al., In vitro effects of the CCR5 inhibitor maraviroc on human T cell function, J. ANTIMICROB. CHEMOTHER., 68(3): 577-586 (2013).

Recently, the functional state of T cells has been characterized by the chemokine receptor expression pattern. In particular, chemokine receptor CCR5 is a marker for effector T cells. CCR5 is a co-receptor for HIV entry, and has been studied extensively. The expression of CCR5 is very low on naïve T cells, but is highly upregulated on both CD4⁺(Th1) T cells and activated antigen specific CD8⁺ T cells.

The function of CCR5 and its ligands in GVHD has been primarily explored in murine models. It has been reported that CCR5⁺/CD8⁺ T cells mediate hepatic injury in mouse GVHD and blocking antibody to CCR5 reduces the damage. In addition, MIP-1α, one of the ligands for CCR5, has also been shown to mediate mouse GVHD. While two groups demonstrated that genetic deletion of CCR5 in the donor can reduce acute GVHD in mice; others reported that CCR5^{-/-} or MIP-1α^{-/-} donor T cells accelerate GVHD in liver and lung. These data suggested that the role of CCR5 in alloimmune responses is complicated, and probably regulated in strain-, target organ- or pretransplant conditioning-dependent fashion in murine GVHD models.

In addition to T cells, other cell types such as dendritic cells (DCs) and B cells are involved in the pathogenesis of GVHD. CCR5 has been shown to express on dendritic cells and specifically the dermal Langerhans cells. Langerhans cells represent the specialized DCs of the epidermis, and play an important role in skin GVHD. However, reports have indicated that the absence of donor expression of CCR5 on T cells ameliorates GVHD in models using no conditioning of the recipient. (See M. Murai, H. Yoneyama, A. Harada, Z. Yi, C. Vestergaard, B. Guo, K. Suzuki, H. Asakura, K. Matsushima, Active participation of CCR5(+)CD8(+) T lymphocytes in the pathogenesis of liver injury in graft-versus-host disease, 104 J. CLIN. INVEST. 49-57 (July 1999).)

Subsequent work assessed the role of CCR5 on donor cells in models with intensive conditioning of the recipient, in an attempt to more accurately mirror the clinical experience. These studies led to the conclusion that the role of CCR5 in allogeneic bone marrow transplants and GVHD is more complex than initially thought. In a murine transplant model with intensive conditioning, the overall effect of absent CCR5 expression on donor cells results in greater GVHD and donor T cell expansion. (See Lisbeth A. Welniak, Zhao Wang, Kai Sun, William Kuziel, Miriam R. Anver, Bruce R. Blazar, William J. Murphy, An absence of CCR5 on donor cells results in acceleration of acute graft-vs-host disease, 32 EXPERIMENTAL HEMATOLOGY 318-324, ISSUE 3 (March 2004).)

Further work on this approach was temporarily abandoned until, in part, a report of the “Berlin patient” cured of HIV with bone marrow (BM) transplant was made. The outcome of allogeneic stem-cell transplantation in a patient with HIV infection and acute myeloid leukemia, using a transplant from an HLA-matched, unrelated donor who was screened for homozygosity for the CCR5 delta32 deletion, indicated a potential cure for AIDS. This experiment also provided insights on GVHD. The patient did not experience severe GVHD. Except for the presence of grade I graft-versus-host disease of the skin, which was treated by adjusting the dosage of cyclosporine, there were no serious infections or toxic effects higher than grade I during the first year of follow-up. (See Gero Hütter, M.D., Daniel Nowak, M.D., Maximilian Mossner, B.S., Susanne Ganepola, M.D., Arne Müßig, M.D., Kristina Allers, Ph.D., Thomas Schneider, M.D., Ph.D., Jörg Hofmann, Ph.D., Claudia Kücherer, M.D., Olga Blau, M.D., Igor W. Blau, M.D., Wolf K. Hofmann, M.D., and Eckhard Thiel, M.D., Long-Term Control of HIV by CCR5 Delta32/Delta32 Stem-Cell Transplantation, 360 N. ENG. J. MED. 692-698 (2009).) Clearly the role of CCR5 is intricate and complicated and the field is still unresolved regarding the role of CCR5 in human GVHD.

Mice rendered genetically suitable to support human cells and tissues have become a favorite model bridging the gap between mouse models and studies in humans (2009, Legrand et al., Cell Host Microbe 6:5-9; 2007, Shultz et al., Nat Rev Immunol 7:118-130; 2007, Manz, Immunity 26:537-541). Particularly, mice that reconstitute a functional human immune system after engraftment of hematopoietic stem and progenitor cells (HSPCs) are of high interest to study GVHD, vaccine candidates, and the biology of pathogens restricted to humans in vivo, as well as immune function generally.

To achieve efficient xenotransplantation, mice lacking an adaptive immune system and natural killer (NK) cells have been successfully developed in the last years, and the major models differ mainly in the background strains used. The first one employs the BALB/c Rag2⁻/⁻yc^{-/-} (DKO) mice, and neonatal intrahepatic HSPC transfer (2004, Traggiai et al., Science 304:104-107; 2004, Gimeno et al., Blood 104:3886-3893). A second model reconstitutes instead NOD/scid/yc−/− (NSG) mice by i.v. or intrahepatic injection of human HSPCs (2002, Ito et al., Blood 100:3175-3182; 2005, Ishikawa et al., Blood 106:1565-1573; 2005, Shultz et al., J Immunol 174:6477-6489). After transfer into these mice, human HSPCs can develop into most of the hematopoietic lineages and the human chimerism is maintained for several months (2004, Traggiai et al., Science 304:104-107; 2005, Ishikawa et al., Blood 106:1565-1573). Overall the composition of engrafted cells is similar in these models, but higher human engraftment levels were obtained in NOD-based models (2010, Brehm et al., Clin Immunol 135:84-98).

Humanized mouse model utility has been enhanced by the development of new stocks of immunodeficient hosts, and mouse strains such as NOD-scid IL2ry null mice that lack the IL-2 receptor common gamma chain. These stocks of mice lack adaptive immune function, display multiple defects in innate immunity, and support heightened levels of human hematolymphoid engraftment. Pearson et. al., Creation of “Humanized” Mice to Study Human Immunity, CURR. PROTOC. IMMUNOL. 2008 May; Chapter: Unit—15.21, doi: 10.1002/0471142735.im1521s81. That is, the NOD-scid IL2ry null strain (NSG) which lacks T- B- and NK-cells, permits acceptance of human tissues and are easily engrafted by human peripheral blood (PB) or bone marrow (BM) derived cells. Shultz L D, Ishikawa F, Greiner D L (2007) Humanized mice in translational biomedical research, NAT REV IMMUNOL 7: 118-130; Shultz L D, Brehm M A, Bavari S, Greiner D L (2011) Humanized mice as a preclinical tool for infectious disease and biomedical research. ALM N Y ACAD SCI 1245: 50-54.

As severely immunocompromised mice lacking T cells, B cells, and NK cells have become widely used hosts for the xenotransplantation of human cells due to their diminished rejection of cells and tissues of human origin, efforts have continued to improve mouse strains, models, and related methodologies to better simulate human immune function. (2004, Traggiai et al., Science 304:104-107; 2002, Ito et al., Blood 100:3175-3182; 2005, Ishikawa et al., Blood 106:1565-1573; 2005, Shultz et al., J Immunol 174:6477-6489).

For example, several approaches have been used to improve human cell engraftment. These include transient approaches such as hydrodynamic injection of plasmid DNA (2009, Chen et al., Proc Natl Acad Sci USA 106:21783-21788), injections of cytokines, and infections of mice or CD34+ cells with lentiviruses (2010, O'Connell et al., PLoS ONE 5:e12009; 2009, Huntington et al., J Exp Med 206:25-34; 2009, van Lent et al., J Immunol 183:7645-7655). Alternatively, transgenic expression of human MHC molecules has been demonstrated to improve the development of antigen-specific immune responses in vivo (2009, Jaiswal et al., PLoS ONE 4:e7251; 2009, Strowig et al., J Exp Med 206:1423-1434; 2011, Danner et al., PLoS ONE 6:e19826). Nonetheless, overexpression of cytokines might also have detrimental side effects due to the unphysiological expression such as in mice transgenic for GM-CSF, and IL-3 (2004, Nicolini et al., Leukemia 18:341-347). Alternatively, human growth factors have been provided in vivo by genetically engineering mice to replace the mouse genes with their human counterparts, resulting in their expression in the appropriate niche at physiological levels. Indeed, faithful replacement of mouse GM-CSF and IL-3 as well as thrombopoietin (TPO) group is reported to have resulted in improved development of human macrophages in the lung, and HSPC and HPC maintenance in the bone marrow, respectively (2011, Rongvaux et al., Proc Natl Acad Sci USA 94:5320-5325; 2011, Willinger et al., Proc Natl Acad Sci USA 108:2390-2395). Notably, in human TPO knockin mice, despite a highly increased engraftment level of stem and progenitor cells in the bone marrow, no changes were observed in the periphery, demonstrating the potential existence of limiting factors in the periphery such as destruction by phagocytes.

There exists a need for competitive inhibitors to the CCR5 receptor and methods of use that can be used to inhibit, dampen, interrupt, block, alter, or modify the CCR5/CCL5 receptor/ligand axis for therapeutic purposes without triggering, or that reduce the impact of, unintended side effects or downstream activities. Further, there is a need for such competitive inhibitors to the CCR5 receptor and methods of use that cause fewer and less severe side effects, are longer-lasting, and facilitate improved patient compliance due to decreased dosing demands and improved patient experience due to fewer undesirable side effects, including such side effects caused by the competitive inhibitor itself. Optimal therapeutic modalities using the CCL5/CCR5 axis as a therapeutic target will need to accommodate two opposing demands: the need to inhibit the detrimental involvement of CCL5 and CCR5 in specific malignant diseases, disorders, or inflammatory conditions, and including GVHD, while protecting their potentially beneficial activities in immunity.

Here, the present inventor provides for the use of binding agents for CCR5 cell receptors, which are known to modulate human immune function, to treat or prevent GVHD in NSG mice. According to the present invention, engrafted subjects or subjects at risk for GVHD are provided with a CCR5 binding agent such as, for example, PRO 140, to treat or prevent GVHD.

### BRIEF SUMMARY

PRO 140 was found to be unexpectedly and profoundly effective in treating and preventing GVHD in NOD.Cg-Prkdc^{scid}Il2ry^{tm1Wjl}/SzJ (NOD-scid IL2ry^{null}, NSG) mice. In certain aspects, the present disclosure is directed to the use of competitive inhibitors of the CCR5 receptor, such as the monoclonal antibody PRO 140, or binding fragments thereof, in the treatment or prevention of GVHD. In additional embodiments, the present invention relates to the use of anti-CCR5 binding agents as single active agents or in combination with other anti-CCR5 binding agents or in combination with one or more other GVHD therapies.

### BRIEF DESCRIPTION OF THE SEVERAL VIEWS OF THE DRAWINGS

FIG. 1 shows the effect of PRO 140 on the mean weight in grams of eight (8) xeno-GVHD in NSG mice administered 2 mg PRO 140 intraperitoneally twice a week (starting on day 1). On day (−1) male NSG mice received 2.25 cGy total body irradiation. On day 0 mice received 10⁷ fresh Ficoll-Hypaque-purified normal human bone marrow cells (56 year old male donor) i.v. via tail vein. Control mice received normal human IgG.

FIG. 2 shows the effect of PRO 140 on the survival of eight (8) xeno-GVHD in NSG mice administered 2 mg PRO 140 intraperitoneally twice a week (starting on day 1). On day(−1) male NSG mice received 2.25 cGy total body irradiation. On day 0 mice received 10⁷ fresh Ficoll-Hypaque-purified normal human bone marrow cells (56 year old male donor) i.v. via tail vein. Control mice received normal human IgG. Percentage (%) survival was analyzed by the Kaplan-Meier method and Mantel-Cox log-rank test.

FIG. 3 shows the effect of PRO 140 on the mean weight in grams of eight (8) xeno-GVHD in NSG mice administered 0.2 mg PRO 140 intraperitoneally twice a week (starting on day 1). On day (−1) male NSG mice received 2.25 cGy total body irradiation. On day 0 mice received 10⁷ fresh Ficoll-Hypaque-purified normal human bone marrow cells i.v. via tail vein. Control mice received normal human IgG.

FIG. 4 shows the effect of PRO 140 on the survival of eight (8) xeno-GVHD in NSG mice administered 0.2 mg PRO 140 intraperitoneally twice a week (starting on day 1). On day(−1) male NSG mice received 2.25 cGy total body irradiation. On day 0 mice received 10⁷ fresh Ficoll-Hypaque-purified normal human bone marrow cells i.v. via tail vein. Control mice received normal human IgG. Percentage (%) survival was analyzed by the Kaplan-Meier method and Mantel-Cox log-rank test.

FIGS. 5A, 5B, 5C, and 5D show the effect of PRO 140 on xeno-GvHD in NSG mice. Flow cytometry analysis of engrafted human cells in peripheral blood from PRO 140 dosed i.p. twice/week started on day 1. Peripheral blood (100 uL) was drawn on the days indicated from the saphenous vein into heparinized tubes. There were 8 animals per group and the experiments were performed twice. The left panels represent the high dose (2.0 mg) experiment (FIG. 5A and FIG. 5C) and the right panels represent the low dose (0.2 mg) experiment (FIG. 5B and FIG. 5D).

FIG. 6 shows the effect of PRO 140 on xeno-GVHD in eight (8) NSG mice administered 2 mg PRO 140 intraperitoneally twice a week (starting on day 1). The graph provides a flow cytometry analysis of engrafted human cells in peripheral blood and in bone marrow on day 54. Peripheral blood (100 uL) was drawn on day 54 from the saphenous vein into heparinized tubes. Human antibodies were used to detect CD45+ cells (all differentiated hematopoietic cells). Peripheral blood (PB) and spleen (SPL) p<0.05, BM N.S. There were 8 animals per group (two experimental groups, control IgG and PRO 140). The top three panels are representative of a single mouse from the control IgG group. The bottom three panels are representative of a single mouse from the PRO 140 group. Asterisks next to the absolute cell counts indicate P<0.05 between experimental groups of eight mice.

FIG. 7 shows the engraftment of human bone marrow (BM) in NSG mice. Human antibodies detect CD45+ cells (all differentiated hematopoietic cells, PE-Cy7 fluorochrome) and CD3 (mature T cells, FITC). The image provides a flow cytometry analysis of gated white blood cells in human donor and murine recipient cells before engraftment (top panels, left is donor bone marrow and right is recipient peripheral blood) and a representative murine recipient of PRO 140 high dose experiment at time of euthanasia, day 75 (bottom panels left peripheral blood and right, bone marrow).

FIGS. 8A and 8B show the effect of PRO 140 on the % of human CD4+ cells and xeno-GVHD in eight (8) NSG mice administered 2 mg PRO 140 intraperitoneally twice a week (starting on day 1) (FIG. 8A) and in eight (8) NSG mice administered 0.2 mg PRO 140 intraperitoneally twice a week (starting on day 1) (FIG. 8B). The graph provides a flow cytometry analysis of engrafted human cells in peripheral blood, spleen, and bone marrow at the time of euthanasia.

### DETAILED DESCRIPTION

The instant disclosure provides methods and compositions for treating or preventing GVHD comprising administering a competitive inhibitor to a CCR5 cell receptor. In some embodiments, the competitive inhibitor comprises PRO 140, or a binding fragment thereof.

### Glossary

Prior to setting forth this disclosure in more detail, it may be helpful to an understanding thereof to provide definitions of certain terms to be used herein. Unless defined otherwise, all technical and scientific terms used herein have the same meaning as is commonly understood by one of skill in the art to which this invention belongs. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as dose, are to be understood to include any integer within the recited range, unless otherwise indicated. As used herein, the term “about” means ±20% of the indicated range, value, or structure, unless otherwise indicated.

It should be understood that the terms “a” and “an” as used herein refer to “one or more” of the enumerated components. The use of the alternative (e.g., “or”) should be understood to mean either one, both, or any combination thereof of the alternatives.

As used herein, the terms “include,” “have,” and “comprise” are used synonymously, which terms and variants thereof are intended to be construed as non-limiting.

The term “consisting essentially of” limits the scope of a claim to the specified materials or steps, or to those that do not materially affect the basic characteristics of a claimed invention. For example, a protein domain, region, or module (e.g., a binding domain, hinge region, linker module) or a protein (which may have one or more domains, regions, or modules) “consists essentially of” a particular amino acid sequence when the amino acid sequence of a domain, region, or module or protein includes extensions, deletions, mutations, or any combination thereof (e.g., amino acids at the amino- or carboxy-terminus or between domains) that, in combination, contribute to at most 20% (e.g., at most 15%, 10%, 8%, 6%, 5%, 4%, 3%, 2%, or 1%) of the length of a domain, region, or module or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “chemokine” means a cytokine that can stimulate leukocyte movement. Chemokines may be characterized as either cys-cys or cys-X-cys depending on whether the two amino terminal cysteine residues are immediately adjacent or separated by one amino acid. It includes but is not limited to CCL5 (also known as RANTES), MIP-1α, MIP-1β, or SDF-1, or another chemokine which has similar activity.

As used herein, “chemokine receptor” means a member of a homologous family of seven-transmembrane spanning cell surface proteins that bind chemokines.

As used herein, “CCR5” is a chemokine receptor which binds members of the C-C group of chemokines and whose amino acid sequence comprises that provided in Genbank Accession Number 1705896, and related polymorphic variants.

As used herein, “antibody” means an immunoglobulin molecule comprising two heavy chains and two light chains and that recognizes an antigen. The immunoglobulin molecule may derive from any of the commonly known classes or isotypes, including but not limited to IgA, secretory IgA, IgG, and IgM. IgG subclasses are also well known to those in the art and include but are not limited to human IgG1, IgG2, IgG3, and IgG4. It includes, by way of example, both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. Optionally, an antibody can be labeled with a detectable marker. Detectable markers include, for example, radioactive or fluorescent markers. The antibody may be a human or nonhuman antibody. The nonhuman antibody may be humanized by recombinant methods to reduce its immunogenicity in humans. Methods for humanizing antibodies are known to those skilled in the art.

As used herein, “monoclonal antibody,” also designated as “mAb,” is used to describe antibody molecules whose primary sequences are essentially identical and which exhibit the same antigenic specificity. Monoclonal antibodies may be produced by hybridoma, recombinant, transgenic, or other techniques known to one skilled in the art.

As used herein, “heavy chain” means the larger polypeptide of an antibody molecule composed of one variable domain (VH) and three or four constant domains (CH1, CH2, CH3, and CH4), or fragments thereof.

As used herein, “light chain” means the smaller polypeptide of an antibody molecule composed of one variable domain (VL) and one constant domain (CL), or fragments thereof.

As used herein, a “binding fragment” or an “antigen-binding fragment or portion” of an antibody refers to the fragment or portion of an intact antibody that has or retains the ability to bind to the antigen target molecule recognized by the intact antibody, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, tetrabodies, tandem di-scFv, and tandem tri-scFv.

As used herein, “Fab” means a monovalent antigen binding fragment of an immunoglobulin that consists of one light chain and part of a heavy chain. It can be obtained by brief papain digestion or by recombinant methods.

As used herein, “F(ab′)2 fragment” means a bivalent antigen binding fragment of an immunoglobulin that consists of both light chains and part of both heavy chains. It can be obtained by brief pepsin digestion or recombinant methods.

As used herein, “CDR” or “complementarity determining region” means a highly variable sequence of amino acids in the variable domain of an antibody.

As used herein, “humanized” describes antibodies wherein some, most or all of the amino acids outside the CDR regions are replaced with corresponding amino acids derived from human immunoglobulin molecules. In one embodiment of the humanized forms of the antibodies, some, most, or all of the amino acids outside the CDR regions have been replaced with amino acids from human immunoglobulin molecules but where some, most, or all amino acids within one or more CDR regions are unchanged. Small additions, deletions, insertions, substitutions, or modifications of amino acids are permissible as long as they would not abrogate the ability of the antibody to bind a given antigen. Suitable human immunoglobulin molecules would include IgG1, IgG2, IgG3, IgG4, IgA, and IgM molecules. A “humanized” antibody would retain a similar antigenic specificity as the original antibody, e.g., in the present disclosure, the ability to bind CCR5.

One skilled in the art would know how to make the humanized antibodies of the subject invention. Various publications, several of which are hereby incorporated by reference into this application, also describe how to make humanized antibodies. For example, the methods described in U.S. Pat. No. 4,816,567 comprise the production of chimeric antibodies having a variable region of one antibody and a constant region of another antibody. U.S. Pat. No. 5,225,539 describes another approach for the production of a humanized antibody. This patent describes the use of recombinant DNA technology to produce a humanized antibody wherein the CDRs of a variable region of one immunoglobulin are replaced with the CDRs from an immunoglobulin with a different specificity such that the humanized antibody would recognize the desired target but would not be recognized in a significant way by the human subject's immune system. Specifically, site directed mutagenesis is used to graft the CDRs onto the framework.

Other approaches for humanizing an antibody are described in U.S. Pat. Nos. 5,585,089 and 5,693,761 and WO 90/07861, which describe methods for producing humanized immunoglobulins. These have one or more CDRs and possible additional amino acids from a donor immunoglobulin and a framework region from an accepting human immunoglobulin. These patents describe a method to increase the affinity of an antibody for the desired antigen. Some amino acids in the framework are chosen to be the same as the amino acids at those positions in the donor rather than in the acceptor. Specifically, these patents describe the preparation of a humanized antibody that binds to a receptor by combining the CDRs of a mouse monoclonal antibody with human immunoglobulin framework and constant regions. Human framework regions can be chosen to maximize homology with the mouse sequence. A computer model can be used to identify amino acids in the framework region which are likely to interact with the CDRs or the specific antigen and then mouse amino acids can be used at these positions to create the humanized antibody.

The above U.S. Pat. Nos. 5,585,089 and 5,693,761 and WO 90/07861 also propose four possible criteria which may be used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid residue at the framework positions at which the amino acid is predicted to have a side chain atom within 3A of the CDRs in a three dimensional model of the antibody, and is predicted to be capable of interacting with the CDRs. The above methods are merely illustrative of some of the methods that one skilled in the art could employ to make humanized antibodies. The affinity and/or specificity of the binding of the humanized antibody may be increased using methods of directed evolution as described in Wu et al., J. MOL. BIOL., 284:151 (1999) and U.S. Pat. Nos. 6,165,793; 6,365,408; and 6,413,774.

The variable regions of the humanized antibody may be linked to at least a portion of an immunoglobulin constant region of a human immunoglobulin. In one embodiment, the humanized antibody contains both light chain and heavy chain constant regions. The heavy chain constant region usually includes CH1, hinge, CH2, CH3, and, sometimes, CH4 region. In one embodiment, the constant regions of the humanized antibody are of the human IgG4 isotype.

The antibodies, or binding fragments, disclosed herein may either be labeled or unlabeled. Unlabeled antibodies can be used in combination with other labeled antibodies (second antibodies) that are reactive with a humanized antibody, such as antibodies specific for human immunoglobulin constant regions. Alternatively, the antibodies can be directly labeled. A wide variety of labels can be employed, such as radionuclides, fluors, enzymes, enzyme substrates, enzyme cofactors, enzyme inhibitors, ligands (particularly haptens), etc. Numerous types of immunoassays are available and are well known to those skilled in the art for detection of CCR5-expressing cells or detection of CCR5 modulation on cells capable of expressing CCR5.

The present disclosure also provides antibody or antibody fragment-polymer conjugates having an effective size or molecular weight that confers an increase in serum half-life, an increase in mean residence time in circulation (MRT), and/or a decrease in serum clearance rate over underivatized antibody fragments. Antibody fragment-polymer conjugates can be made by derivatizing the desired antibody fragment with an inert polymer. It will be appreciated that any inert polymer which provides the conjugate with the desired apparent size, or which has the selected actual molecular weight, is suitable for use in constructing antibody fragment-polymer conjugates of the invention.

Many inert polymers are suitable for use in pharmaceuticals. See, e.g., Davis et al., Biomedical Polymers: Polymeric Materials and Pharmaceuticals for Biomedical Use, pp. 441-451 (1980). For the antibody or antibody fragment-polymer conjugates disclosed herein, a non-proteinaceous polymer is used. The nonproteinaceous polymer ordinarily is a hydrophilic synthetic polymer, i.e., a polymer not otherwise found in nature. However, polymers which exist in nature and are produced by recombinant or in vitro methods are also useful, as are polymers which are isolated from native sources. Hydrophilic polyvinyl polymers fall within the scope of this invention, e.g., polyvinyl alcohol and polyvinylpyrrolidone. Particularly useful are polyalkylene ethers such as polyethylene glycol (PEG); polyoxyalklyenes such as polyoxyethylene, polyoxypropylene, and block copolymers of polyoxyethylene and polyoxypropylene (Pluronics); polymethacrylates; carbomers; branched or unbranched polysaccharides which comprise the saccharide monomers D-mannose, D- and L-galactose, fucose, fructose, D-xylose, L-arabinose, D-glucuronic acid, sialic acid, D-galacturonic acid, D-mannuronic acid (e.g., polymannuronic acid, or alginic acid), D-glucosamine, D-galactosamine, D-glucose, and neuraminic acid including homopolysaccharides and heteropolysaccharides such as lactose, amylopectin, starch, hydroxyethyl starch, amylose, dextran sulfate, dextran, dextrins, glycogen, or the polysaccharide subunit of acid mucopolysaccharides, e.g., hyaluronic acid, polymers of sugar alcohols such as polysorbitol and polymannitol, heparin, or heparon. The polymer prior to cross-linking need not be, but preferably is, water soluble, but the final conjugate must be water soluble. Preferably, the conjugate exhibits a water solubility of at least about 0.01 mg/ml and more preferably at least about 0.1 mg/ml, and still more preferably at least about 1 mg/ml. In one embodiment, the polymer should not be highly immunogenic in the conjugate form, nor should it possess viscosity that is incompatible with intravenous infusion or injection if the conjugate is intended to be administered by such routes.

In one embodiment, the polymer contains only a single group, which is reactive. This helps to avoid cross-linking of protein molecules. However it is within the scope of the invention to maximize reaction conditions to reduce cross-linking, or to purify the reaction products through gel filtration or ion-exchange chromatography to recover substantially homogeneous derivatives. In other embodiments, the polymer contains two or more reactive groups for the purpose of linking multiple antibody fragments to the polymer backbone.

Gel filtration or ion-exchange chromatography can be used to recover the desired derivative in substantially homogeneous form.

The molecular weight of the polymer can range up to about 500,000 D and preferably is at least about 20,000 D, or at least about 30,000 D, or at least about 40,000 D. The molecular weight chosen can depend upon the effective size of the conjugate to be achieved, the nature (e.g., structure such as linear or branched) of the polymer and the degree of derivatization, i.e., the number of polymer molecules per antibody fragment, and the polymer attachment site or sites on the antibody fragment.

The polymer can be covalently linked to the antibody fragment through a multifunctional crosslinking agent which reacts with the polymer and one or more amino acid residues of the antibody fragment to be linked. However, it is also within the scope of the invention to directly crosslink the polymer by reacting a derivatized polymer with the antibody fragment, or vice versa.

The covalent crosslinking site on the antibody fragment includes the N-terminal amino group and epsilon amino groups found on lysine residues, as well other amino, imino, carboxyl, sulfhydryl, hydroxyl, or other hydrophilic groups. The polymer may be covalently bonded directly to the antibody fragment without the use of a multifunctional (ordinarily bifunctional) crosslinking agent, as described in U.S. Pat. No. 6,458,355.

The degree of substitution with such a polymer will vary depending upon the number of reactive sites on the antibody fragment, the molecular weight, hydrophilicity and other characteristics of the polymer, and the particular antibody fragment derivatization sites chosen. In general, the conjugate contains from 1 to about 10 polymer molecules, but greater numbers of polymer molecules attached to the antibody fragments of the invention are also contemplated. The desired amount of derivatization is easily achieved by using an experimental matrix in which the time, temperature, and other reaction conditions are varied to change the degree of substitution, after which the level of polymer substitution of the conjugates is determined by size exclusion chromatography or other means known in the art.

Functionalized PEG polymers to modify the antibody fragments of the invention are available from Shearwater Polymers, Inc. (Huntsville, Ala.). Such commercially available PEG derivatives include, but are not limited to, amino-PEG, PEG amino acid esters, PEG-hydrazide, PEG-thiol, PEG-succinate, carboxymethylated PEG, PEG-propionic acid, PEG amino acids, PEG succinimidyl succinate, PEG succinimidyl propionate, succinimidyl ester of carboxymethylated PEG, succinimidyl carbonate of PEG, succinimidyl esters of amino acid PEGs, PEG-oxycarbonylimidazole, PEG-nitrophenyl carbonate, PEG tresylate, PEG-glycidyl ether, PEG-aldehyde, PEG-vinylsulfone, PEG-maleimide, PEG-orthopyridyl-disulfide, heterofunctional PEGs, PEG vinyl derivatives, PEG silanes, and PEG phospholides. The reaction conditions for coupling these PEG derivatives will vary depending on the protein, the desired degree of PEGylation, and the PEG derivative utilized. Some factors involved in the choice of PEG derivatives include: the desired point of attachment (such as lysine or cysteine R-groups), hydrolytic stability and reactivity of the derivatives, stability, toxicity and antigenicity of the linkage, suitability for analysis, etc. Specific instructions for the use of any particular derivative are available from the manufacturer. The conjugates of which may be separated from the unreacted starting materials by gel filtration or ion exchange HPLC.

As used herein, “anti-chemokine receptor antibody” means an antibody which recognizes and binds to an epitope on a chemokine receptor. As used herein, “anti-CCR5 antibody” means a monoclonal antibody that recognizes and binds to an epitope on the CCR5 chemokine receptor.

As used herein, “epitope” means a portion of a molecule or molecules that forms a surface for binding antibodies or other compounds. The epitope may comprise contiguous or noncontiguous amino acids, carbohydrate, or other nonpeptidyl moieties or oligomer-specific surfaces.

As used herein, “polypeptide” means two or more amino acids linked by a peptide bond.

A “nucleic acid molecule,” or “polynucleotide,” may be in the form of RNA or DNA, which includes cDNA, genomic DNA, and synthetic DNA. A nucleic acid molecule may be double stranded or single stranded, and if single stranded, may be the coding strand or non-coding (anti-sense strand). A coding molecule may have a coding sequence identical to a coding sequence known in the art or may have a different coding sequence, which, as the result of the redundancy or degeneracy of the genetic code, or by splicing, can encode the same polypeptide.

“Analogs” of antibodies or binding fragments include molecules differing from the antibodies or binding fragments by conservative amino acid substitutions. For purposes of classifying amino acid substitutions as conservative or nonconservative, amino acids may be grouped as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Nonconservative substitutions constitute exchanging a member of one of these classes for a member of another.

Due to the degeneracy of the genetic code, a variety of nucleic acid sequences encode the proteins or polypeptides disclosed herein. For example, homologous nucleic acid molecules may comprise a nucleotide sequence that is at least about 90% identical to a reference nucleotide sequence. More preferably, the nucleotide sequence is at least about 95% identical, at least about 97% identical, at least about 98% identical, or at least about 99% identical to a reference nucleotide sequence. The homology can be calculated using various, publicly available software tools well known to one of ordinary skill in the art. Exemplary tools include the BLAST system available from the website of the National Center for Biotechnology Information (NCBI) at the National Institutes of Health.

One method of identifying highly homologous nucleotide sequences is via nucleic acid hybridization. Thus, homologous nucleic acid molecules hybridize under high stringency conditions. Identification of related sequences can also be achieved using polymerase chain reaction (PCR) and other amplification techniques suitable for cloning related nucleic acid sequences. Preferably, PCR primers are selected to amplify portions of a nucleic acid sequence of interest, such as a CDR.

The term “high stringency conditions” as used herein refers to parameters with which the art is familiar. Nucleic acid hybridization parameters may be found in references that compile such methods, e.g., MOLECULAR CLONING: A LABORATORY MANUAL, J. Sambrook, et al., eds., Second Edition, Cold Spring Harbor Laboratory Press, Cold Spring Harbor, N.Y., (1989), or CURRENT PROTOCOLS IN MOLECULAR BIOLOGY, F. M. Ausubel, et al., eds., John Wiley & Sons, Inc., New York. One example of high stringency conditions is hybridization at 65 degrees Centigrade in hybridization buffer (3.5×SSC, 0.02% Ficoll, 0.02% polyvinyl pyrrolidone, 0.02% Bovine Serum Albumin, 2.5 mM NaH2PO4 (pIl7), 0.5% SDS, 2 mM EDTA). SSC is 0.15 M sodium chloride/0.015 M sodium citrate, pH7; SDS is sodium dodecyl sulphate; and EDTA is ethylenediaminetetracetic acid. After hybridization, a membrane upon which the nucleic acid is transferred is washed, for example, in 2×SSC at room temperature and then at 0.1-0.5×SSC/0.1×SDS at temperatures up to 68 degrees Centigrade.

As used herein, the term “vector” refers to a nucleic acid molecule that is capable of transporting another nucleic acid. Vectors may be, for example, plasmids, cosmids, viruses, or phage. An “expression vector” is a vector that is capable of directing the expression of a protein encoded by one or more genes carried by the vector when it is present in the appropriate environment.

Nucleic acid sequences may be expressed in hosts after the sequences have been operably linked to (i.e., positioned to ensure the functioning of) an expression control sequence. These expression vectors are typically replicable in the host organisms, either as episomes or as an integral part of the host chromosomal DNA. Commonly, expression vectors will contain selection markers, e.g., tetracycline or neomycin, to permit detection of those cells transformed with the desired DNA sequences. See, e.g., U.S. Pat. No. 4,704,362, which is incorporated herein by reference.

E. coli is one prokaryotic host useful for cloning the DNA sequences of the present invention. Other microbial hosts suitable for use include bacilli, such as Bacillus subtilus, and other enterobacteriaccae, such as Salmonella, Serratia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation.

Other microbes, such as yeast, may also be useful for expression. Saccharomyces is a preferred host, with suitable vectors having expression control sequences, such as promoters, including 3-phosphoglycerate kinase or other glycolytic enzymes and an origin of replication, termination sequences, and the like as desired.

In addition to microorganisms, mammalian tissue cell culture may also be used to express and produce the polypeptides of the present invention. See Winnacker, From Genes to Clones, VCH Publishers, New York, N.Y. (1987). Eukaryotic cells are actually preferred, because a number of suitable host cell lines capable of secreting intact immunoglobulins have been developed in the art, and include the CHO cell lines, various COS cell lines, HeLa cells, preferably myeloma cell lines, etc., and transformed B cells or hybridomas. Expression vectors for these cells can include expression control sequences, such as an origin of replication, a promoter, an enhancer (Queen et al., IMMUNOL. REV., 89: 49-68 (1986), which is incorporated herein by reference), and necessary processing information sites, such as ribosome binding sites, RNA splice sites, polyadenylation sites, and transcriptional terminator sequences. Preferred expression control sequences are promoters derived from immunoglobulin genes, SV40, Adenovirus, cytomegalovirus, Bovine Papilloma Virus, and the like.

The vectors containing the DNA segments of interest (e.g., the heavy and light chain encoding sequences and expression control sequences) can be transferred into the host cell by well-known methods, which vary depending on the type of cellular host. For example, calcium chloride transfection is commonly utilized for prokaryotic cells, whereas calcium phosphate treatment or electroporation may be used for other cellular hosts. See generally, Maniatis et al., MOLECULAR CLONING: A LABORATORY MANUAL, Cold Spring Harbor Press (1982), which is incorporated herein by reference.

Once expressed, the whole antibodies, their dimers, individual light and heavy chains, or other immunoglobulin forms or binding fragments of the present invention, can be purified according to standard procedures of the art, including ammonium sulfate precipitation, affinity columns, column chromatography, gel electrophoresis, and the like. See generally, R. Scopes, PROTEIN PURIFICATION, Springer-Verlag, New York (1982). Substantially pure immunoglobulins of at least about 90 to 95% homogeneity are preferred, and 98 to 99% or more homogeneity most preferred, for pharmaceutical uses. Once purified, partially or to homogeneity as desired, the polypeptides may then be used therapeutically (including extracorporeally) or in developing and performing assay procedures, immunofluorescent stainings, and the like. See generally, IMMUNOLOGICAL METHODS, Vols. I and II, Lefkovits and Pernis, eds., Academic Press, New York, N.Y. (1979 and 1981).

As used herein, “inhibits” means that the amount is reduced in the presence of a composition as compared with the amount that would occur without the composition.

The term “competitive inhibitor” as used herein refers to a molecule that competes with a reference molecule for binding to a target, and thereby blunts, inhibits, dampens, reduces, or blocks the effects of the reference molecule on the target. For example, PRO 140 is a competitive inhibitor of CCL5 binding to CCR5 receptor.

“Agonist activity” as used in the present disclosure refers to the binding by a molecule to a target, wherein the binding activates the target to produce a response.

“CCL5 agonist activity,” as used herein, refers to activity consistent with activation by CCL5.

“Antagonist activity” as used in the present disclosure refers to the binding by a molecule to a target, wherein the binding does not activate the target to produce a response and the binding blocks the action of one or more agonist molecules.

As used herein, “subject” means any animal or artificially modified animal capable of having cancer. Artificially modified animals include, but are not limited to, SCID mice with human immune systems. The animals include but are not limited to mice, rats, dogs, guinea pigs, ferrets, rabbits, and primates. In a preferred embodiment, the subject is a human.

As used herein, “treating” means slowing, stopping, or reversing the progression of a given disease or disorder. In a preferred embodiment, “treating” means reversing the progression of the disease or disorder. In some embodiments, treating includes reversing the progression of the disease or disorder to the point of eliminating the disease or disorder.

As used herein, “preventing” refers to preventing a disease or disorder from occurring; delaying the progression of a disease or disorder; or reducing the pathology or symptomatology of a disease or disorder. For example, preventing a GVHD includes preventing the development of GVHD, slowing the growth of GVHD, and delaying the development of GVHD.

As used herein, “administering” may be effected or performed using any of the methods known to one skilled in the art. The methods may comprise intravenous, intramuscular, or subcutaneous means.

As used herein, “effective dose” means an amount in sufficient quantities to either treat the subject or prevent the subject from developing GVHD. A person of ordinary skill in the art can perform simple titration experiments to determine what amount is required to treat the subject.

### The CCR5 Cell Receptor

As described above, the CCR5 cell receptor, or CCR5 receptor, is important in many immune responses. It is likely that CCR5 plays a role in inflammatory responses to infection, although its exact role in normal immune function is not completely defined.

The CCR5 receptor is a C-C chemokine G-coupled protein receptor expressed on lymphocytes (e.g., NK cells, B cells), monocytes, macrophages, dendritic cells, a subset of T cells, etc. CCR5 is predominantly expressed on T cells, macrophages, dendritic cells, eosinophils and microglia. The CCR5 protein belongs to the beta chemokine receptor family of integral membrane proteins. CCR5-chemokine (C-C motif) receptor 5 (gene/pseudogene). Genetics Home Reference (“CCR5-chemokine”); Samson M, Labbe O, Mollereau C, Vassart G, Parmentier M (1996) Molecular cloning and functional expression of a new human CC-chemokine receptor gene, BIOCHEMISTRY 35: 3362-3367.

The CCR5 receptor spans the cellular plasma membrane seven times in a serpentine manner. The extracellular portions represent potential targets for therapeutic agents, including antibodies, targeting CCR5, and comprise an amino-terminal domain (Nt) and three extracellular loops (ECL1, ECL2, and ECL3). The extracellular portions of CCR5 comprise just 90 amino acids distributed over four domains. The largest of these domains are at the Nt and ECL2 at approximately 30 amino acids each. Olson et al., CCR5 Monoclonal Antibodies for HIV-1 Therapy, CURR. OPIN. HIV AIDS, March, 4(2): 104-111 (2009). Regions of this protein are crucial for chemokine ligand binding, functional response of the receptor, and also HIV co-receptor activity. Barmania F, Pepper M S (2013), C-C CHEMOKINE RECEPTOR TYPE FIVE (CCRS): AN EMERGING TARGET FOR THE CONTROL OF HIV INFECTION, APPLIED & TRANSLATIONAL GENOMICS 2: 3-16.

Chemokines bind to receptors expressed on many cell types, including, for example, leukocytes, endothelial cells, fibroblasts, epithelial, smooth muscle, and parenchymal cells. Chemokines play an important role in leukocyte biology, by controlling cell recruitment and activation in basal and in inflammatory circumstances. Because chemokine receptors are expressed on other cell types, chemokines have multiple other roles, including angiogenesis, tissue and vascular remodeling, pathogen elimination, antigen presentation, leukocyte activation and survival, chronic inflammation, tissue repair/healing, fibrosis, embryogenesis, tumorigenesis, etc.

The CCR5 receptor's cognate ligands include CCL5 (RANTES), CCL3, CCL4 (also known as MIP 1 a and 1/1, respectively), and CCL3L1. Struyf S, Menten P, Lenaerts J P, Put W, D'Haese A, De Clercq E, et al. (2001), Diverging binding capacities of natural LD78beta isoforms of macrophage inflammatory protein-lalpha to the CC chemokine receptors 1, 3, and 5 affect their anti-HIV-1 activity and chemotactic potencies for neutrophils and eosinophils, EUROPEAN JOURNAL OF IMMUNOLOGY 31: 2170-2178; Miyakawa T, Obaru K, Maeda K, Harada S, Mitsuya H (2002), Identification of amino acid residues critical for LD78beta, a variant of human macrophage inflammatory protein-I alpha, binding to CCR5 and inhibition of RS human immunodeficiency virus type 1 replication, THE JOURNAL OF BIOLOGICAL CHEMISTRY 227: 4649-4655. CCL5, or RANTES, is a chemotactic cytokine protein. Struyf; Slimani H, Charnaux N, Mbemba E, Saffar L, Vassay R, Vita C, et al. (2003), Interaction of RANTES with syndecan-1 and syndecan-4 expressed by human primary macrophages, BIOCHEMICA ET BIOPHYSICAACTA 1617: 80-88 (“Slimani”); Barmania F, Pepper M S (2013), C-C CHEMOKINE RECEPTOR TYPE FIVE (CCRS): AN EMERGING TARGET FOR THE CONTROL OF HIV INFECTION, APPLIED & TRANSLATIONAL GENOMICS 2: 3-16 (“Barmania”).

The formation of the CCL5 ligand and CCR5 receptor complex causes a conformational change in the receptor that activates the subunits of the G-protein, inducing signaling and leading to changed levels of cyclic AMP (cAMP), inositol triphosphate, intracellular calcium, and tyrosine kinase activation. These signaling events cause cell polarization and translocation of the transcription factor NF-kB, which results in the increase of phagocytic ability, cell survival, and transcription of proinflammatory genes. Once G-protein dependent signaling occurs, the CCL5/CCR5 receptor complex is internalized via endocytosis.

A complete complex structure of CCL5 in complex with CCR5 has been computationally derived. It is reported that the 1-15 residue moiety of CCL5 is inserted into the CCR5 binding pocket; the 1-6 N-terminal domain of CCL5 is buried within the transmembrane region of CCR5; and the 7-15 residue moiety of CCL5 is predominantly encompassed by the N-terminal domain and extracellular loops of CCR5. CCL5 residues Ala16 and Arg17 and additional residues of the 24-50 residue moiety interact with the upper N-terminal domain and extracellular loop interface of CCR5. It is further reported that the integrity of the amino terminus of CCL5 is crucial to receptor binding and cellular activation. Further, it has been reported that CCL5 and HIV-1 primarily interact with mostly the same CCR5 residues, and share the same chemokine receptor binding pocket. See Tamamis et al., Elucidating a Key Anti-HIV-1 and Cancer-Associated Axis: The Structure of CCL5 (Rantes) in Complex with CCR5, SCIENTIFIC REPORTS, 4:5447 (2014). It is also separately reported that chemokines, such as the CCL5 ligand, principally bind the CCR5 receptor through ECL2. Olson et al., CCR5 Monoclonal Antibodies for HIV-1 Therapy, CURR. OPIN., HIV AIDS, March, 4(2): 104-111 (2009).

### Non-Chemokine CCR5 Cell Receptor Binding Agents

CCR5+s exact role in normal immune function is not completely defined. But CCR5 seems to have a broad effect on this process as it has been described to mediate the recruitment of effector T cells, as well as Tregs, to many different target organs. Boieri et al., The Role of Animal Models in the Study of Hematopoietic Stem Cell Transplantation and GvHD: A Historical Overview, FRONTIERS IN IMMUNOLOGY, Aug. 2016 7:333. Accordingly, blocking chemokine—chemokine receptor interaction is a therapeutic strategy that has been tested using animal models. Administration of anti-CXCR3 or anti-CX3CL1 antibodies in mouse models of aGVHD were shown to reduce gastrointestinal aGVHD. However, targeting CCR5 has given contrasting results as this chemokine is thought to be involved also in Treg recruitment to peripheral tissues. Id.

Various compounds exist that inhibit, interrupt, block, alter, or modify the CCR5/CCL5 receptor/ligand axis (i.e., CCR5 receptor/CCL5 ligand axis). Many of these compounds have been developed for the treatment of HIV-1, which also binds with the CCR5 receptor and is known to share some binding commonalities with CCL5. Such compounds include extracellular or cell transmembrane CCR5 binding agents such as, for example, PRO 140 (extracellular) and maraviroc (transmembrane), and other compounds such as vicriviroc, aplaviroc, SCH-C, and TAK-779, and antibodies such as PA14, 2D7, RoAb13, RoAb14, 45523, etc.

In one embodiment, the present disclosure provides for the use of a PRO 140 antibody, or binding fragment thereof, in treating or preventing GVHD. PRO 140 is a humanized monoclonal antibody described in US Pat. Nos. 7,122,185 and 8,821,877, which are incorporated herein by reference, in their entirety. PRO 140 is a humanized version of the murine mAb, PA14, which was generated against CD4⁺ CCR5⁺ cells. Olson et al., Differential Inhibition of Human Immunodeficiency Virus Type 1 Fusion, gp 120 Binding and CC-Chemokine Activity of Monoclonal Antibodies to CCR5, J. VIROL., 73: 4145-4155. (1999). PRO 140 binds to CCR5 expressed on the surface of a cell, and potently inhibits HIV-1 entry and replication at concentrations that do not affect CCR5 chemokine receptor activity in vitro and in the hu-PBL-SCID mouse model of HIV-1 infection. Olson et al., Differential Inhibition of Human Immunodeficiency Virus Type 1 Fusion, gp 120 Binding and CC-Chemokine Activity of Monoclonal Antibodies to CCR5, J. VIROL., 73: 4145-4155. (1999); Trkola et al., Potent, Broad-Spectrum Inhibition of Human Immunodeficiency Virus Type 1 by the CCR5 Monoclonal Antibody PRO 140, J. VIROL., 75: 579-588 (2001).

Nucleic acids encoding heavy and light chains of the humanized PRO 140 antibody have been deposited with the ATCC. Specifically, the plasmids designated pVK-HuPRO140, pVg4-HuPRO140 (mut B+D+I) and pVg4-HuPRO140 HG2, respectively, were deposited pursuant to, and in satisfaction of, the requirements of the Budapest Treaty with the ATCC, Manassas, Va., U.S.A. 20108, on Feb. 22, 2002, under ATCC Accession Nos. PTA 4097, PTA 4099, and PTA 4098, respectively.

Inhibition of CCR5 signaling has also been shown to have immunosuppressive effects. For example, in vitro studies have been conducted to investigate the effects of CCR5 receptor blockade by maraviroc on activated human T cells on potential immunological mechanisms. It was found that blocking CCR5 by maraviroc not only can block CCR5 and CCR2 internalization processes induced by CCL5 and CCL2, but can also inhibit T cell chemotactic activities toward their cognate ligands, respectively. Further, blocking CCR5 with maraviroc at high doses tends to decrease production of TNF-α and IFN-γ. It was also noted that the effect of maraviroc on CCR5 was temporary and reversible. Yuan et al., In Vitro Immunological Effects of Blocking CCR5 on T Cells, INFLAMMATION, 38(2): 902-910 (2015); see Arberas et al., In vitro effects of the CCR5 inhibitor maraviroc on human T cell function, J. ANTIMICROB. CHEMOTHER., 68(3): 577-586 (2013).

PRO 140 binds to the CCR5 receptor and was developed as an entry inhibitor for HIV, has completed seven clinical trials as an investigative HIV therapeutic entity, and is currently in two FDA approved Phase 2b/3 clinical trials for HIV infection. Specifically, PRO 140 is a competitive inhibitor of CCR5 with binding reactivity to the second external loop of CCR5. Olson W C, Rabut G E E, Nagashima K A, Tran D N H, Anselma D J, Monard S P, et al. (1999), Differential inhibition of human immunodeficiency virus type 1 fusion, gp120 binding, and CC-chemokine activity by monoclonal antibodies to CCRS, JOURNAL OF VIROLOGY 73: 4145-4155 (“Olson”). Importantly, PRO 140 binding to CCR5 has been shown to not result in CCL5 ligand (RANTES) agonist activities, and may dampen such activities, as assessed by downstream triggering of cAMP or tyrosine kinase activity; however, PRO 140 but does not appear to dampen certain other downstream effects resulting from cell exposure to RANTES. See PCT/US2016/039016.

In a one embodiment, the methods disclosed herein comprise administering a humanized antibody designated PRO 140 or an antibody that competes with PRO 140 for binding to the CCR5 receptor, wherein the PRO 140 comprises (i) two light chains, each light chain comprising the expression product of the plasmid designated pVK:HuPRO140-VK (ATCC Deposit Designation PTA-4097), and (ii) two heavy chains, each heavy chain comprising the expression product of either the plasmid designated pVg4:HuPRO140 HG2-VH (ATCC Deposit Designation PTA-4098) or the plasmid designated pVg4:HuPRO140 (mut B+D+I)-VH (ATCC Deposit Designation PTA-4099). In a further embodiment, the PRO 140 is a humanized or human antibody that binds to the same epitope as that to which antibody PRO 140 binds. In another embodiment, the monoclonal antibody is the humanized antibody designated PRO 140.

In a further embodiment, the present disclosure relates to the use of the human antibody designated CCR5mAb004, or a binding fragment thereof. CCR5mAb004 is a fully human mAb, generated using the Abgenix XenoMouse® technology, that specifically recognizes and binds to CCR5. See Roschke et al., Characterization of a Panel of Novel Human Monoclonal Antibodies That Specifically Antagonize CCR5 and Block HIV Entry, 44th Annual Interscience CONFERENCE ON ANTIMICROBIAL AGENTS AND CHEMOTHERAPY, Washington, D.C., Oct. 30-Nov. 2, 2004 (2004); HGS Press Release, Human Genome Sciences Characterizes Panel of Novel Human Monoclonal Antibodies That Specifically Antagonize the CCR5 Receptor and Block HIV-1 Entry, Nov. 2, 2004 (2004); HGS Press Release, Human Genome Sciences Begins Dosing of Patients in a Phase 1 Clinical Trial of CCR5 mAb in Patients Infected With HIV-1, Mar. 30, 2005 (2005).

In one embodiment, the present disclosure relates to the use of the monoclonal antibody PA14, produced by the hybridoma cell line designated PA14 (ATCC Accession No. HB-12610), a binding fragment thereof, or an antibody that competes with monoclonal antibody PA-14 in binding to the CCR5 receptor, in treating or preventing GVHD.

In one embodiment of the methods described herein, the antibody or binding fragment thereof comprises a light chain of the antibody. In another embodiment, the antibody or binding fragment thereof comprises a heavy chain of the antibody. In a further embodiment, the antibody or binding fragment thereof comprises an Fab portion of the antibody. In a still further embodiment, the antibody or binding fragment thereof comprises an F(ab')₂ portion of the antibody. In an additional embodiment, the antibody or binding fragment thereof comprises an Fd portion of the antibody. In another embodiment, the antibody or binding fragment thereof comprises an Fv portion of the antibody. In a further embodiment, the antibody or binding fragment thereof comprises a variable domain of the antibody. In a still further embodiment, the antibody or binding fragment thereof comprises one or more CDR domains of the antibody. In yet another embodiment, the antibody or binding fragment thereof comprises six CDR domains of the antibody.

In other embodiments, the method of treating or preventing GVHD include the use of one or more anti-CCR5 binding agents, including antibodies or fragments thereof, other peptides, or small molecules.

### Methods of Use

In one aspect, the present disclosure provides methods of treating or preventing GVHD comprising administering to a subject in need thereof a competitive inhibitor to a CCR5 cell receptor that does not itself have CCL5 agonist activity. In certain embodiments, the competitive inhibitor to a CCR5 cell receptor does not itself have CCL5 agonist activity in terms of affecting cAMP levels when added to CD4⁺ T cells or chemotaxis of CHO-K1 cells. In certain embodiments, the competitive inhibitor to a CCR5 cell receptor does not itself have CCL5 agonist activity in terms of affecting tyrosine kinase phosphorylation downstream of CCR5 receptor signaling in T cells in vitro, and also does not inhibit phosphorylation of such kinases by CCL5. In a particular embodiment, a method for preventing GVHD is provided.

In one embodiment, the present disclosure provides a method of preventing GVHD comprising administering to a subject in need thereof a competitive inhibitor to a CCR5 cell receptor that does not itself have CCL5 agonist activity, and wherein the competitive inhibitor binds to the ECL-2 loop of the CCR5 cell receptor. In a further embodiment, the competitive inhibitor competes with CCL5 for binding to the CCR5 cell receptor. In a further embodiment, the competitive inhibitor comprises the monoclonal antibody PA14, PRO 140, or CCR5mAb004, or a binding fragment thereof. In a further embodiment, the competitive inhibitor competes for binding with the monoclonal antibody PA14, PRO 140, or CCR5mAb004, or a binding fragment thereof.

In one embodiment, the present disclosure provides a method of preventing GVHD comprising administering to a subject in need thereof: (a) a PRO 140 antibody, or binding fragment thereof; (b) a nucleic acid encoding a PRO 140 antibody, or binding fragment thereof; (c) a vector comprising a nucleic acid encoding a PRO 140 antibody, or binding fragment thereof; (d) a host cell comprising (i) a PRO 140 antibody, or binding fragment thereof, (ii) a nucleic acid encoding a PRO 140 antibody, or binding fragment thereof, or (iii) a vector comprising a nucleic acid encoding a PRO 140 antibody, or binding fragment thereof; or (e) another peptide. In the aforementioned embodiment, the PRO 140 antibody, or binding fragment thereof, may comprise, for example, a PRO 140 monoclonal antibody or a scFv.

In one embodiment, the present disclosure provides a method of treating or preventing GVHD comprising administering to a subject in need thereof a PRO 140 antibody, or binding fragment thereof.

In any of the aforementioned embodiments, preventing GVHD may comprise slowing the growth of the GVHD, preventing the formation of GVHD, or limiting or reducing the symptoms associated with GVHD.

In a preferred embodiment, formation of GVHD is completely prevented in allotransplant subjects administered with PRO 140 or a fragment thereof. The PRO 140 may be administered before transplant, during transplant, or after transplant. In a particularly preferred embodiment, the subject is treated with PRO 140 before transplant. In another preferred embodiment, the subject is treated with PRO 140 as soon as possible upon or immediately subsequent to transplant.

In one embodiment, the competitive inhibitor to a CCR5 cell receptor, such as PRO 140, is administered with a pharmaceutically acceptable carrier. Pharmaceutically acceptable carriers are well known to those skilled in the art. Such pharmaceutically acceptable carriers may include but are not limited to aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, saline, and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like.

The disclosure of U.S. patent application Ser. No. 13/582,243 relates to concentrated protein formulations and particularly discloses formulations of PRO 140, and is incorporated by reference here.

The dose of the composition of the invention will vary depending on the subject and upon the particular route of administration used. Dosages can range from 0.1 μg/kg to 100,000 μg/kg. In another embodiment the antibody or binding fragment thereof is formulated to deliver between 100 mg/mL to 200 mg/mL of the antibody or binding fragment thereof to the subject. In another embodiment the antibody or binding fragment thereof is formulated to deliver between 100 mg/mL to 150 mg/mL of the antibody or binding fragment thereof to the subject. In another embodiment the antibody or binding fragment thereof is formulated to deliver between 150 mg/mL to 200 mg/mL of the antibody or binding fragment thereof to the subject. In another embodiment the antibody or binding fragment thereof is formulated to deliver 175 mg/mL of the antibody or binding fragment thereof to the subject. Based upon the composition, the dose can be delivered continuously, such as by continuous pump, or at periodic intervals, e.g., on one or more separate occasions. Desired time intervals of multiple doses of a particular composition can be determined without undue experimentation by one skilled in the art.

In one embodiment of the instant methods, the antibody or binding fragment thereof is administered to the subject a plurality of times, and each administration delivers from 0.01 mg per kg body weight to 50 mg per kg body weight of the antibody or binding fragment thereof to the subject. In another embodiment, each administration delivers from 0.05 mg per kg body weight to 25 mg per kg body weight of the antibody or binding fragment thereof to the subject. In a further embodiment, each administration delivers from 0.1 mg per kg body weight to 10 mg per kg body weight of the antibody or binding fragment thereof to the subject. In a still further embodiment, each administration delivers from 0.5 mg per kg body weight to 5 mg per kg body weight of the antibody or binding fragment thereof to the subject. In another embodiment, each administration delivers from 1 mg per kg body weight to 3 mg per kg body weight of the antibody or binding fragment thereof to the subject. In a another embodiment, each administration delivers about 2 mg per kg body weight of the antibody or binding fragment thereof to the subject.

In a preferred embodiment, the antibody or binding fragment thereof is administered in a formulation comprising 175 mg/mL of the anti-CCR5 binding agent, and may be delivered in two 1 mL shots for administration of about 350 mg in total to a subject in need thereof.

In one embodiment, the antibody or binding fragment thereof is administered a plurality of times, and a first administration is separated from the subsequent administration by an interval of less than one week. In another embodiment, the first administration is separated from the subsequent administration by an interval of at least one week. In a further embodiment, the first administration is separated from the subsequent administration by an interval of one week. In another embodiment, the first administration is separated from the subsequent administration by an interval of two to four weeks. In another embodiment, the first administration is separated from the subsequent administration by an interval of two weeks. In a further embodiment, the first administration is separated from the subsequent administration by an interval of four weeks. In yet another embodiment, the antibody or binding fragment thereof is administered a plurality of times, and a first administration is separated from the subsequent administration by an interval of at least one month.

In a further embodiment, the antibody or binding fragment thereof is administered to the subject via intravenous infusion. In another embodiment, the antibody or binding fragment thereof is administered to the subject via subcutaneous injection. In another embodiment, the antibody or binding fragment thereof is administered to the subject via intramuscular injection.

In one embodiment, the aforementioned methods may further comprise administering to the subject a cellular therapy, e.g., an autologous or allogeneic immunotherapy; a small molecule; a chemotherapeutic agent; or an inhibitor of CCR5/CCL5 signaling. In one embodiment, an inhibitor of CCR5/CCL5 signaling is administered, and comprises maraviroc, vicriviroc, aplaviroc, SCH-C, TAK-779, PA14 antibody, 2D7 antibody, RoAb13 antibody, RoAb14 antibody, or 45523 antibody.

In one embodiment, the competitive inhibitor to a CCR5 cell receptor, such as PRO 140, is administered in combination with one or more other therapeutic molecules or treatment, such a cellular therapy, e.g., an autologous or allogeneic immunotherapy; a small molecule; a chemotherapeutic; or an inhibitor of CCR5/CCL5 signaling, such as maraviroc, vicriviroc, aplaviroc, SCH-C, TAK-779, PA14 antibody, 2D7 antibody, RoAb13 antibody, RoAb14 antibody, or 45523 antibody. In one embodiment, the methods disclosed herein comprise administering PRO 140 in combination with maraviroc, vicriviroc, aplaviroc, SCH-C, TAK-779, PA14 antibody, 2D7 antibody, RoAb13 antibody, RoAb14 antibody, or 45523 antibody.

As used herein, a “small-molecule” CCR5 receptor antagonist includes, for example, a small organic molecule which binds to a CCR5 receptor and inhibits the activity of the receptor. In one embodiment, the small molecule has a molecular weight less than 1,500 daltons. In another embodiment, the small molecule has a molecular weight less than 600 daltons.

In one embodiment, the competitive inhibitor to a CCR5 cell receptor, such as PRO 140, is administered in combination with one or more small molecules, such as SCH-C (Strizki et al., PNAS, 98: 12718-12723 (2001)); SCH-D (SCH 417670; vicriviroc); UK-427,857 (maraviroc; 1-[(4,6-dimethyl-5-pyrimidinyl) carbonyl]-4-[4-[2-methoxy-1(R)-4-(trifluoromethyl)phenyl]ethyl-3(S)-methyl-1-piperazinyl]-4-methylpiperidine); GW873140; TAK-652; TAK-779; AMD070; AD101; 1,3,4-trisubstituted pyrrolidines (Kim et al., BIOORG. MED. CHEM. LETT., 15: 2129-2134 (2005)); modified 4-piperidinyl-2-phenyl-1-(phenylsulfonylamino)-butanes (Shah et al., BIOORG. MED. CHEM. LETT., 15: 977-982 (2005)); Anibamine TFA, Ophiobolin C, or 19,20-epoxycytochalasin Q (Jayasuriya et al., J. NAT. PROD., 67: 1036-1038 (2004)); 5-(piperidin-1-yl)-3-phenyl-pentylsulfones (Shankaran et al., BIOORG. MED. CHEM. LETT., 14: 3589-3593 (2004)); 4-(heteroarylpiperdin-1-yl-methyl)-pyrrolidin-1-yl-acetic acid antagonists (Shankaran et al., BIOORG. MED. CHEM. LETT., 14: 3419-3424 (2004)); agents containing 4-(pyrazolyl)piperidine side chains (Shu et al., BIOORG. MED. CHEM. LETT., 14: 947-52 (2004); Shen et al., BIOORG. MED. CHEM. LETT., 14: 935-939 (2004); Shen et al., BIOORG. MED. CHEM. LETT., 14: 941-945 (2004)); 3-(pyrrolidin-1-yl)propionic acid analogues (Lynch et al., Org. Lett., 5: 2473-2475 (2003)); [2-(R)-[N-methyl-N-(1-(R)-3-(S)-((4-(3-benzyl-1-ethyl-(1H)-pyrazol-5-yl)piperidin-1-yl)methyl)-4-(S)-(3-fluorophenyl)cyclopent-1-yl)amino]-3-methylbutanoic acid (MRK-1)] (Kumar et al., J. PHARMACOL. EXP. THER., 304: 1161-1171 (2003)); 1,3,4 trisubstituted pyrrolidines bearing 4-aminoheterocycle substituted piperidine side chains (Willoughby et al., BIOORG. MED. CHEM. LETT., 13: 427-431 (2003); Lynch et al., BIOORG. MED. CHEM. LETT., 12: 3001-3004 (2003); Lynch et al., BIOORG. MED. CHEM. LETT., 13: 119-123 (2003); Hale et al., BIOORG. MED. CHEM. LETT., 12: 2997-3000 (2002)); bicyclic isoxazolidines (Lynch et al., BIOORG. MED. CHEM. LETT., 12: 677-679 (2002)); combinatorial synthesis of CCR5 antagonists (Willoughby et al., BIOORG. MED. CHEM. LETT., 11: 3137-41 (2001)); heterocycle-containing compounds (Kim et al., BIOORG. MED. CHEM. LETT., 11: 3103-3106 (2001)); antagonists containing hydantoins (Kim et al., BIOORG. MED. CHEM. LETT., 11: 3099-3102 (2001)); 1,3,4 trisubstituted pyrrolidines (Hale et al., BIOORG. MED. CHEM. LETT., 11: 2741-2745 (2001)); 1-[N-(methyl)-N-(phenylsulfonyl)amino]-2-(phenyl)-4-(4- (N-(alkyl)-N-(benzyloxycarbonyl)amino)piperidin-1-yl)butanes (Finke et al., BIOORG. MED. CHEM. LETT., 11: 2475-2479 (2001)); compounds from the plant Lippia alva (Hedge et al., BIOORG. MED. CHEM. LETT., 12: 5339-5342 (2004)); piperazine-based CCR5 antagonists (Tagat et al., J. MED. CHEM., 47: 2405-2408 (2004)); oximino-piperidino-piperidine-based CCR5 antagonists (Palani et al., BIOORG. MED. CHEM. LETT., 13: 709-712 (2003)); rotamers of SCH 351125 (Palani et al., BIOORG. MED. CHEM. LETT., 13: 705-708 (2003)); piperazine-based symmetrical heteroaryl carboxamides (McCombie et al., BIOORG. MED. CHEM. LETT., 13: 567-571 (2003)); oximino-piperidino-piperidine amides (Palani et al., J. MED. CHEM., 45: 3143-3160 (2002)); Sch-351125 and Sch-350634 (Este, CURR. OPIN. INVESTIG. DRUGS., 3: 379-383 (2002)); 1-[(2,4-dimethyl-3-pyridinyl)carbonyl]-4-methyl-4-[3(S)-methyl-4-[1(S)[4-(trifluoromethyl)phenyl]ethyl]-1-piperazinyl]-piperidine N1-oxide (Sch-350634) (Tagat et al., J. MED. CHEM., 44: 3343-3346 (2001)); 4-[(Z)-(4-bromophenyl)-(ethoxyimino)methyl]-1′-[(2,4-dimethyl-3-pyridinyl)carbonyl]-4′-methyl-1,4′-bipiperidine N-oxide (SCH 351125) (Palani et al., J. MED. CHEM., 44: 3339-3342 (2001)); 2(S)-methyl piperazines (Tagat et al., BIOORG. MED. CHEM. LETT., 11: 2143-2146 (2001)); piperidine-4-carboxamide derivatives (Imamura et al., BIOORG. MED. CHEM., 13: 397-416, 2005); 1-benzazepine derivatives containing a sulfoxide moiety (Seto et al., BIOORG. MED. CHEM. LETT., 13: 363-386 (2005)); anilide derivatives containing a pyridine N-oxide moiety (Seto et al., CHEM. PHARM. BULL. (Tokyo), 52: 818-829 (2004)); 1-benzothiepine 1,1-dioxide and 1-benzazepine derivatives containing a tertiary amine moiety (Seto et al., CHEM. PHARM. BULL. (Tokyo), 52: 577-590 (2004)); N-[3-(4-benzylpiperidin-1-yl)propyl]-N,N′-diphenylureas (Imamura et al., BIOORG. MED. CHEM., 12: 2295-2306 (2004)); 5-oxopyrrolidine-3-carboxamide derivatives (Imamura et al., CHEM. PHARM. BULL. (Tokyo), 52: 63-73 (2004); anilide derivatives with a quaternary ammonium moiety (Shiraishi et al., J. MED. CHEM., 43: 2049-2063 (2000)); AK602/ONO4128/GW873140 (Nakata et al., J. VIROL., 79: 2087-2096 (2005)); spirodiketopiperazine derivatives (Maeda et al., J. BIOL. CHEM., 276: 35194-35200 (2001); Maeda et al., J. VIROL., 78: 8654-8662 (2004)); and selective CCR5 antagonists (Thoma et al., J. MED. CHEM., 47: 1939-1955 (2004)).

In one embodiment, the competitive inhibitor to a CCR5 cell receptor, such as PRO 140, is administered in combination with one or more of SCH-C, SCH-D (SCH 417670, or vicriviroc), UK-427,857 (maraviroc), GW873140, TAK-652, TAK-779 AMD070, or AD101. See U.S. Pat. No. 8,821,877.

In one embodiment, the competitive inhibitor to a CCR5 cell receptor, such as PRO 140, exhibits synergistic effects when administered in combination with one or more other therapeutic molecules or treatment, such as a cellular therapy, a small molecule, a chemotherapeutic, or an inhibitor of CCR5/CCL5 signaling. “Synergy” between two or more agents refers to the combined effect of the agents which is greater than their additive effects. Synergistic, additive, or antagonistic effects between agents may be quantified by analysis of the dose-response curves using the Combination Index (CI) method. A CI value greater than 1 indicates antagonism; a CI value equal to 1 indicates an additive effect; and a CI value less than 1 indicates a synergistic effect. In one embodiment, the CI value of a synergistic interaction is less than 0.9. In another embodiment, the CI value is less than 0.8. In another embodiment, the CI value is less than 0.7.

The anti-CCR5 agent of the present invention may be administered one or more times before, during, or after transplant to treat or prevent GVHD.

Production of Transgenic Non-Human Animals that are Engrafted with a Human Hematopoietic System

Hematopoietic stem cells may be sourced from, for example, bone marrow, peripheral blood, and cord blood.

Generally, two basic protocols describe generating humanized mice: Basic Protocol 1 deals with hematopoietic stem cell (HSC) engraftment (human SCID repopulating cell; hu-SRC) and Basic Protocol 2 addresses engraftment with human peripheral blood mononuclear cells (PBMC) (human peripheral blood leukocyte; hu-PBL). Pearson et. al., Creation of “Humanized” Mice to Study Human Immunity, CURR. PROTOC. IMMUNOL. 2008 May; Chapter: Unit—15.21, doi: 10.1002/0471142735.im1521s81.

The main advantage of the HSC engraftment model (hu-SRC-SCID) is that the human T and B cells develop from human stem cells engrafted in the mouse, undergo negative selection during differentiation into T and B cells, and are therefore tolerant of the mouse host. This model allows for investigation of hematopoietic lineage development and mechanisms of immune system development and the generation of primary immune responses by a naïve immune system. Pearson et. al., Creation of “Humanized” Mice to Study Human Immunity, CURR. PROTOC. IMMUNOL. 2008 May; Chapter: Unit—15.21, doi: 10.1002/0471142735.im1521s81.

The PBMC model (hu-PBL-SCID) utilizes leukocytes isolated from peripheral whole blood or spleen, and allows for rapid analysis of human immune function because the transferred lymphocytes are functionally mature. This model is best suited for studies of immune function from patients with immunologic disorders, analyses of antigen recall responses, investigations of allograft rejection, and other short-term (˜4-week) experiments. Pearson et. al., Creation of “Humanized” Mice to Study Human Immunity, CURR. PROTOC. IMMUNOL. 2008 May; Chapter: Unit—15.21, doi: 10.1002/0471142735.im1521s81.

In many instances, total body irradiation (TBI) prior to engraftment has been a standard conditioning regimen to achieve high levels of human cell engraftment in xenograft animal models because it triggers the secretion of stem cell factor (SCF), which is critical for hematopoietic stem cell engraftment, proliferation, and survival. However, other conditioning regimens, including depletion of mouse macrophages or granulocytes prior to engraftment or administration of chemotherapeutic agents, such as bulsulfan, have been explored. Kang et al., 2016; Pearson, 2008. Additional conditioning regimen efforts to improve engraftment have included, for example, treatment of engrafted mice with human cytokines, or co-engraftment with mesenchymal stem cells. Pearson, 2008.

This disclosure relates to the HSC engraftment model for the generation of chimeras by xeno-transplantation. Accordingly, this model encompasses the administration of human cells or tissue in usually immunodeficient animals. Excellent host animals for generating a human immune system are mouse lines that have several defects in the adaptive immunity such as Rag^{2-/-/}y^{-/-}, BNX or NOD/SCID B2m^{null}.

This disclosure relates to the use of NOD.Cg-Prkdc^{scid}Il2ry^{tm1wjl}/SzJ (NOD-scid IL2ry^{null}, NSG) mice to study GVHD in mice with humanized immune systems.

Different lines of the NOD/SCID (non-obese-diabetic/severe combined immunodeficiency) mouse serve as a standard model for humanization. They are characterized essentially by the following immunodeficiency properties: complete loss of B lymphocytes and T lymphocytes, reduced number of NK cells, defects in the differentiation and function of antigen-presenting cells, and the absence of circulating complement. These mice are more susceptible for ionizing radiation than the wild type, and have defects in the DNA repair system. The formation of human individual lines or several lines of hematopoiesis in an immunodeficient animal is possible after transplantation of human hematopoietic stem cells, differentiated hematopoietic cells, as well as lymphoid organs.

Here, the present inventor found that administration of an anti-CCR5 binding agent to immunodeficient mice provided improved engraftment, in terms of engraftment success, animal health, and animal longevity, using the HSC engraftment model for the generation of chimeras by xeno-transplantation. Specifically, a humanized monoclonal antibody, PRO 140, was administered to NOD-scid IL2ry^{null}, NSG mice upon HSC engraftment. Surprisingly, mice administered PRO 140 exhibited significantly improved health (e.g., weight maintenance and appearance), and longevity (e.g., 100% survival past 70 days in a xeno-transplant animal model), while also demonstrating successful engraftment. Perhaps even more spectacularly, the PRO 140 treated mice of the present invention did not exhibit any symptoms associated with GVHD. That is, administration of PRO 140 to the mice receiving transplanted bone marrow prevented GVHD and, spectacularly, this prevention was complete and uniform in all studied mice.

It is noted that graft-versus-host disease (GVHD) is an exemplary human disease for study by a transgenic non-human animal (here, mouse) engrafted with a human hematopoietic system. Alteration of mouse models used to study GVHD continue to offer insights into the extreme complexities of human immune function generally, and specifically, in this disease pathology.

As new therapeutic options to treat GVHD are badly needed, the humanized mouse models used to study this disease, and related modifications to these models, offer valuable insights into whether and how such modifications to mouse strains, mouse models, and related methodologies may impact humanized mouse immune systems, engraftment, and related human therapeutic options.

GVHD is noted here with particular interest with respect to immune cell trafficking and the inventor's focus on modulation of CCR5 cell receptor binding because GVHD pathophysiology includes migration of lymphocytes to their target tissues as one of the key steps. That is, it is understood that chemokines and chemokine receptors, such as the CCR5 cell receptor, specifically guide T cells in this process.

Accordingly, the present invention is informed by GVHD mouse model with a human transplant and humanized immune system that evidences the utility of the claimed methods and compositions for treating or preventing GVHD comprising administering a competitive inhibitor to a CCR5 cell receptor. In some embodiments, the competitive inhibitor comprises PRO 140, or a binding fragment thereof.

### EXAMPLES

These examples describe the present invention as realized in a GVHD mouse model. As stated elsewhere in this application, GVHD is a prevalent and potentially lethal complication following hematopoietic stem cell transplantation. Humanized mouse models of xenogeneic-GVHD are important tools to evaluate the human immune response in vivo.

It is noted that GVHD can develop, for example, following allogeneic hematopoietic stem cell transplantation (HSCT), which has an important role in a variety of malignant and non-malignant hematological diseases. Donor derived T cell alloreactivity to human leukocyte antigens (HLA) disparities can result in GVHD, which is potentially life threatening. New therapies are needed to address GVHD other than lymphoid depletion strategies, as this non-specific approach leaves patients at risk of complications such as infection or cancer relapse. Champlin R, Ho W, Gajewski J, Feig S, Burnison M, Holley G, et al. (1990), Selective depletion of CD8+ T lymphocytes for prevention of graft-versus-host disease after allogeneic bone marrow transplantation, BLOOD 76: 418-423; Gallardo D, Garcia-Lopez J, Sureda A, Canals C, Ferra C, Cancelas J A, et al. (1997), Low-dose donor CD8+ cells in the CD4-depleted graft prevent allogeneic marrow graft rejection and severe graft-versus-host disease for chronic myeloid leukemia patients in first chronic phase, BONE MARROW TRANSPLANT 20: 945-952.

For example, GVHD in the Hu-SRC-SCID model for NSG mice is dependent on human immune cell xeno-reactivity against mouse Major Histocompatibility Class I and class II antigens (MHC) similar to HLA mismatched HSCT where donor alloreactivity is initiated by recognition of recipient MHC antigens. King M A, Covassin L, Brehm M A, Racki W, Pearson T, Leif J, et al. (2009) Human peripheral blood leucocyte non-obese diabetic-severe combined immunodeficiency interleukin-2 receptor gamma chain gene mouse model ofxenogeneic graft-versus-host-like disease and the role of host major histocompatibility complex. CLIN EXP IMMUNOL 230 157: 104-118; Reddy P, Ferrara J L (2003) Immunobiology of acute graft-versus-host disease. BLOOD REV 17: 187-194. The involvement of specific organs in acute GVHD of HSCT recipients suggests that immune cell trafficking is crucial to the pathophysiology of this disease.

Here, the inventor evaluated PRO 140, a humanized monoclonal antibody which targets a chemokine receptor, C-C chemokine receptor type 5 (CCR5 or CD195) as an inhibitor of the development of xeno-GVHD. Inhibition of lymphocyte trafficking using a CCR5 antagonist has previously been shown to reduce the impact of acute GVHD in patients undergoing HSCT. Reshef R, Luger S M, Hexner E O, Loren A W, Frey N V, Goldstein S C, et al. (2011), Inhibition of lymphocyte trafficking using a CCR5 antagonist—final result of a phase I/II study, BLOOD 118: 1011; Reshef R, Mangan J K, Luger S M, Loren A W, Hexner E O, Frey N Y, et al. (2014), Extended CCR5 blockade in graft-versus-host disease prophylaxis—a phase II study, BLOOD 124: 2491; and Moy R H, Huffman A P, Richman L P, Crisalli L, Wang X K, Hoxie J A, et al. (2017), Clinical and immunologic impact of CCR5 blockade in graft-versus-host disease prophylaxis, BLOOD 129: 906-916.

As discussed below, administration of PRO 140 to the NSG mice following injection of hematopoietic stem cells resulted in a dramatic, significant, and surprising increase in mouse health and survival, a positive GVHD therapeutic effect, and human CD45+ cell engraftment levels after 75 days of greater than about 75% in peripheral blood and greater than about 65% in bone marrow. The contents of inventor's publication, PRO 140 Monoclonal Antibody to CCR5 Prevents Acute Xenogeneic Graft-versus-Host Disease in NOD-scid IL-2Ry^{null} Mice, BIOL. BLOOD MARROW TRANSPLANT, 2018 February; 24(2): 260-266, are hereby incorporated by this reference.

Here NOD-scid IL-2Ry^{null} mice (NSG) were transplanted with human bone marrow cells to evaluate the role of immune cell trafficking in the production of acute GVHD. PRO 140 was used to evaluate its influence on bone marrow cell engraftment and modulation of acute GVHD. Engraftment kinetics were evaluated by assessing human CD45+ cells and CD3+ T cells in treated and control mice. In peripheral blood, spleen and bone marrow, PRO 140 treated mice showed no signs of GVHD throughout the 70-day study period, and gained weight until sacrifice at 70 days for flow cytometry analysis. The control mice started losing weight after 25 days, showed classic signs of GVHD (ruffled fur, lethargy, etc.) and all required sacrifice by Day 54. The percentage of human CD45+ cells in peripheral blood increased in both groups of mice throughout the 50-day comparison period, but was significantly lower in the PRO 140 treated mice at day 50. Importantly, there was no difference in control and PRO 140 treated mice in human CD45+ cells detected in bone marrow at Day 70. By masking the CCR5 chemokine receptor, PRO 140 eliminated acute GVHD in this humanized mouse model without significantly altering engraftment.

### Animal Studies:

Animal experiments were conducted in accordance with the ethical standards and according to national and international guidelines and were approved by the Cleveland Clinic Institutional Animal Care and Use Committee. Male NSG mice, NOD.Cg-Prkdc^{scid}Il2ry^{tm1wjl}/SzJ (NOD-scid IL2ry^{null}, NSG) mice were obtained from the Jackson Laboratory (Bar Harbor, Me., USA), and athymic nude (nu/nu) (Taconic, Hudson, N.Y.) 6-8 wk old were used. Mice were housed in a barrier facility in cages with microisolator lids, autoclaved bedding, and HEPA-filtered air, and maintained under 12:12 light/dark cycles, controlled temperature and humidity. Animals had free access to autoclaved standard food and filtered water. Conditioning regimen: Mice received 2.25 Gy total body irradiation via a 137Cs source (Shepherd, Los Angeles Calif.).

### Bone Marrow Transplantation and Generation of Xeno-GVHD:

Following gamma irradiation (24 h later) mice were engrafted with human BM cells. De-identified human donor cells were obtained by back-flushing filter packs utilized by the Cleveland Clinic BMT program. Fresh (non-frozen) leukocytes were purified by Ficoll-Hypaque gradient centrifugation, washed in phosphate buffered saline (PBS), assessed for viability (ViCell, Beckman Coulter, Brea, Calif.). Human BM leukocytes were injected into the lateral tail vein (107 cells/mouse). Mice were monitored for clinical symptoms of GVHD (body posture, activity, fur and skin condition, weight loss) two times/wk. Peripheral blood was monitored weekly for engraftment utilizing saphenous vein venipuncture (50 mL) collected in K-EDTA tubes. Mice exhibiting 20% weight loss with clinical symptoms of GVHD were considered to have reached experimental endpoint and were subject to euthanasia by controlled gradient CO₂ inhalation.

PRO 140 treatment:

Mice were randomized into control and treatment groups by body weight. PRO 140 was administered on different doses (0.2-2.0 mg/kg) and schedules. The route was always intraperitoneal (i.p.). Control mice received normal human IgG (Sigma Aldrich, St. Louis, Mo.).

PRO 140 dosage was calculated using “Representative Surface Area to Weight Ratios (km) for Various Species” from: Freireich et al., Quantitative comparison of toxicity of anticancer agents in mouse, rat, hamster, dog, monkey, and man, CANCER CHEMOTHER REP., 50:219-44 (1966); and the National Cancer Institute Developmental Therapeutics Program http://dtp.nci.nih.gov. Starting with the human dose of PRO 140=5.8 mg/kg×12 (man-to-mouse conversion factor)=69.6 mg/kg mouse dose; average mouse=0.025 kg, therefore dose is 69.6 mg/kg×0.025 kg=1.74 mg (mouse single dose). This was rounded up to 2.0 mg and designated as the “high dose”. A “low dose” (0.2 mg) was also tested. IgG derived from human serum (>95% SDS-PAGE, Sigma, I4506) was used as a non-specific control antibody.

### Flow Cytometry:

Peripheral blood (PB), bone marrow (BM), and spleen (SPL) samples were analyzed by flow cytometry. Splenocytes were passed through a 40 mm strainer. Erythrocytes were lysed with ammonium chloride. Cells were washed twice with PBS and stained for 15 min at 4 deg C. in PBS/0.5 mM EDTA/0.5% BSA with the following antibodies: anti-human-CD3-FITC (clone UCHT1, IM1281U), anti-human-CD45-PC7 (clone J.33, 1M3548U), Beckman/Coulter. Samples were analyzed on a Cytomics FC500 Flow Analyzer (Beckman/Coulter).

### Statistical Analysis:

Statistical analysis was performed using GraphPad Prism (GraphPad Software, La Jolla, Calif.). All measures of variance were depicted as standard error of the mean (SEM). Survival was analyzed by the Kaplan-Meier method and Mantel-Cox log-rank test. For other data, two-sided unpaired Students t-test was used.

### Results

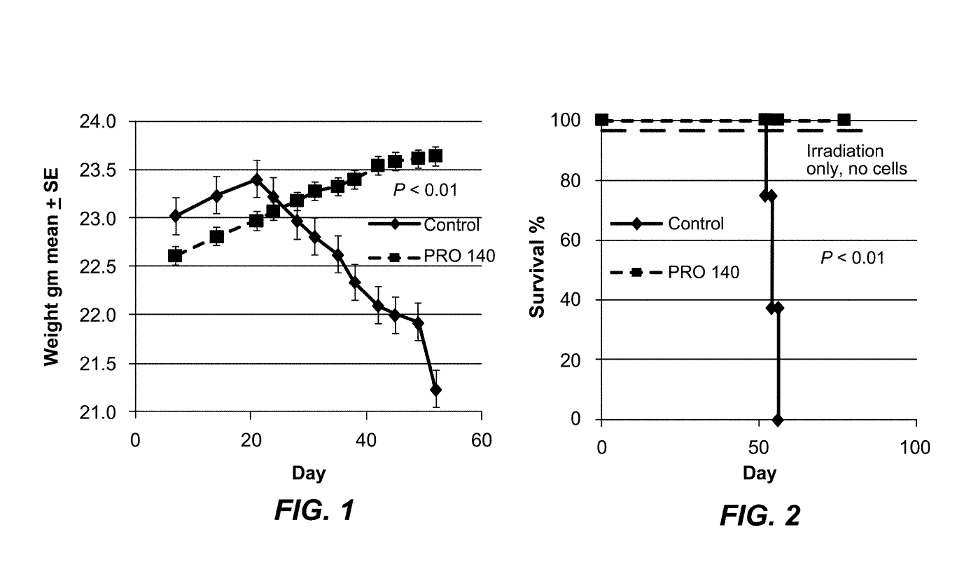

The effects of PRO 140 on the development of acute GVHD was evaluated in the xenogeneic NSG mouse model. The hallmarks of GVHD in NSG mice, including observed physical signs (ruffled fur, lethargy, severe hunching), measured weight loss, organ involvement and death, were assessed. Physical signs of GVHD were observed in control mice (treated with a non-specific IgG) starting at day 25 after engraftment, and included weight loss. Weight loss continued in the control group and was significantly different (p=0.021) from the PRO 140 treated group, which showed no physical signs of GVHD and continued to gain weight (FIG. 1; PRO 140 2.0 mg i.p. twice/wk started day 1). When survival was assessed in a Kaplan-Meier plot (FIG. 2; PRO 140 2.0 mg i.p. twice/wk started day 1), the results were highly statistically significant (p=0.008), with all of the control animals dead by 56 days and all of the PRO 140 treated animals alive at 70 days, when sacrificed for flow cytometry analysis of engraftment.
FIG. 1. Effect of PRO 140 on xeno-GVHD in NSG mice—weight; high dose.

On day (−1) male NSG mice received 2.25 cGy total body irradiation. On day 0 mice received 107 fresh Ficoll-Hypaque-purified normal human bone marrow cells (56 year old male donor) i.v. via tail vein. Control mice received normal human IgG. As can be seen, the control mice starting losing weight at about 20 days after engraftment, and this weight loss continued from a high of about 23.4 gm at about 20 days to about 21.2 gm after about 52 days. Meanwhile the mice in the PRO 140 treated group (PRO 140 2.0 mg i.p. twice/wk started day 1) gained weight over the same time period, going from about 23.0 gm at about 20 days to about 23.6 gm at about 52 days.
FIG. 2. Effect of PRO 140 on xeno-GVHD in NSG mice—survival; high dose.

On day (−1) male NSG mice received 2.25 cGy total body irradiation. On day 0 mice received 107 fresh Ficoll-Hypaque-purified normal human bone marrow cells (56 year old male donor) i.v. via tail vein. Control mice received normal human IgG. As shown in FIG. 2, all of the control animals were dead by 56 days and all of the PRO 140 treated animals (PRO 140 2.0 mg i.p. twice/wk started day 1) were alive at 70 days, when sacrificed for flow cytometry analysis of engraftment.

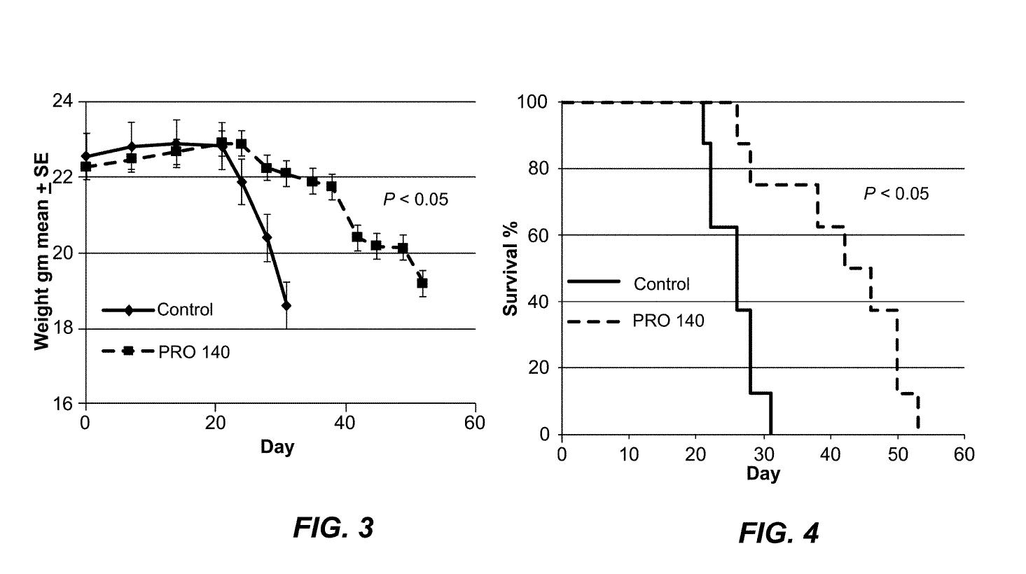

FIG. 3 and FIG. 4 show the effect of PRO 140 on xeno-GVHD in NSG mice at a low 0.2 mg dosing schedule with respect to weight and survivability. On day (−1) male NSG mice received 2.25 cGy total body irradiation. On day 0 mice received 10⁷ fresh Ficoll-Hypaque-purified normal human bone marrow cells (male donor) i.v. via tail vein. Control mice received normal human IgG. On day 1 mice received 0.2 mg PRO 140 i.p. n=8 mice/group.

By necessity, the high and low dose studies were done in succession using different BM donors. In the two sets of experiments, BM donors of different ages were used. Consistent with published data, the younger donor used on the low dose cohort resulted in more aggressive GvHD when time to death was compared (31 days vs. 54 days, FIGS. 2, 4) (Rezvani A R, Storer B E, Guthrie K A, et al. (2015) Impact of donor age on outcome after allogeneic hematopoietic cell transplantation, BIOL BLOOD MARROW TRANSPLANT 21(1): 105-112). The extent to which the lower dose of PRO 140 versus a more aggressive BM contributed separately to the weight loss and the Kaplan-Meier plots was not independently assessed in this study.

In the low dose study using one-tenth the dose, physical signs of GvHD were observed in control mice starting at day 20 after engraftment with BM from a 26-year-old donor and included ruffled fur, lethargy and hunching with weight loss starting shortly thereafter. Weight loss continued in the control group and was significantly different (P<0.05) from the PRO 140-treated group which showed physical signs of GvHD and weight loss starting at day 25-28 (FIG. 2). When survival was assessed in a Kaplan-Meier plot (FIG. 2), the results were statistically significant (P<0.05) with all of the control animals dead by 31 days and all of the PRO 140-treated animals dead by 54 days. As indicated by survival time in control animals from high and low dose studies (54 vs. 31 days), the younger BM donor produced more aggressive GvHD.

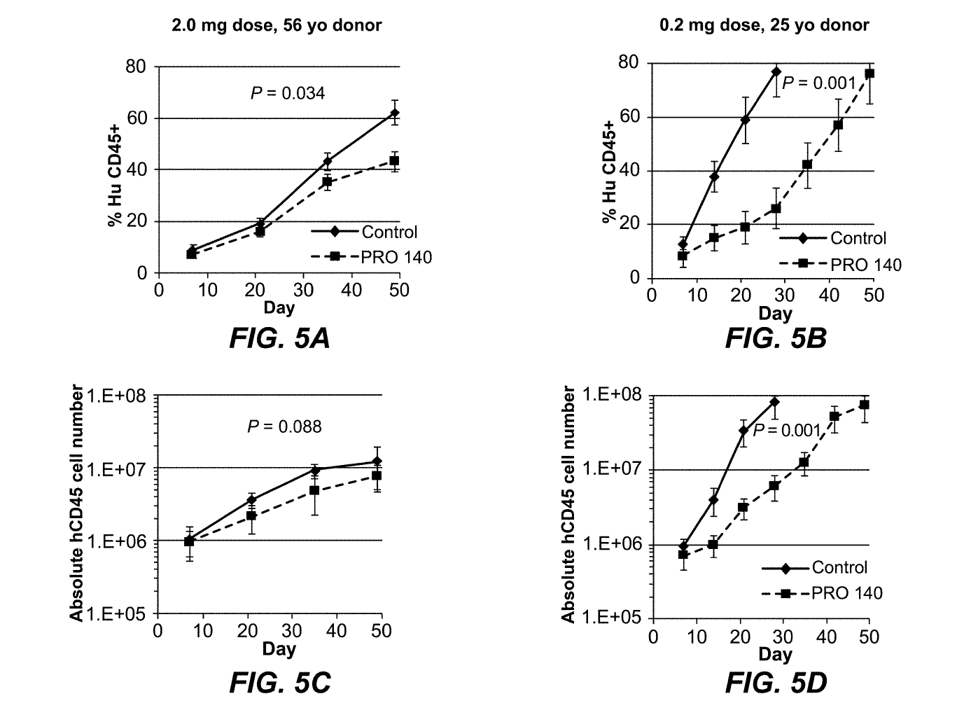

FIGS. 5A, 5B, 5C, and 5D show the effect of PRO 140 on xeno-GvHD in NSG mice. Flow cytometry analysis of engrafted human cells in peripheral blood from PRO 140 dosed i.p. twice/week started on day 1. Peripheral blood (100 uL) was drawn on the days indicated from the saphenous vein into heparinized tubes. There were 8 animals per group and the experiments were performed twice. The left panels represent the high dose (2.0 mg) experiment (FIG. 5A and FIG. 5C) and the right panels represent the low dose (0.2 mg) experiment (FIG. 5B and FIG. 5D).

Analysis of the kinetics of engraftment in the peripheral circulation by flow cytometry using an antibody specific for human CD45+ cells (all differentiated hematopoietic cells), showed similar engraftment for the first 30 plus days (FIGS. 5A, 5B, 5C, and 5D), then diverged with significantly less cells detected in the CD45+ compartment at 50 days in the PRO 140 animals (62% vs. 43%, p=0.034). This is at a time when the control animals are exhibiting severe GVHD. It is contemplated that the PRO 140 reduced inflammation leading to the lower human CD45+ cells count at 50 days. In the low dose cohort, there was a divergence in engraftment starting at 15 days. The same percentage of CD45⁺ engraftment was achieved albeit approximately 20 days later in the low dose PRO 140 treated mice (P<0.01). This observation was supported by a determination of the absolute number of cells in the peripheral circulation during this time frame (FIG. 5C and FIG. 5D).

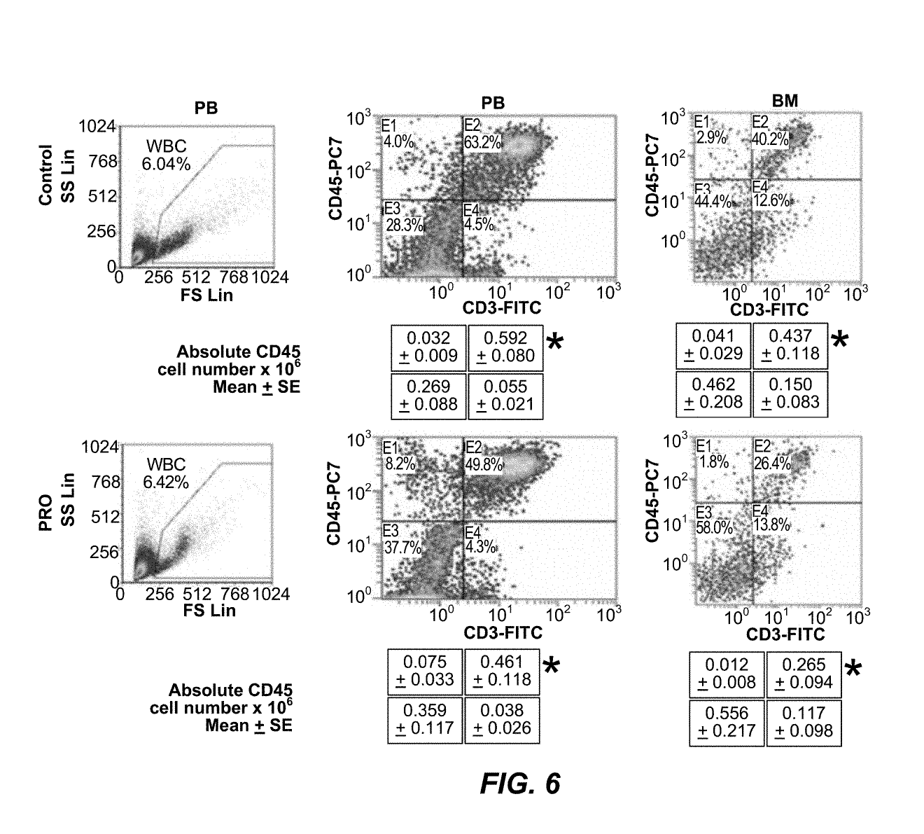

FIG. 6 depicts engraftment of human BM in NSG mice using antibodies to human (hu CD45) and mouse (m CD45) CD45. These antibodies were used to detect the percent of engraftment in bone marrow (BM) and peripheral blood (PB) of PRO 140 treated mice engrafted with human bone marrow cells.

In the high dose cohort at 54 days, analysis of engraftment was assessed in peripheral blood and bone marrow with antibodies specific for CD45 (identifies all differentiated hematopoietic cells) and CD3 (mature T Cells). In PB, the engraftment of mature T-cells was greater in control compared to PRO 140-treated animals (63.2% vs. 49.8%, FIG. 6, PB panels, E2 quadrants). In the BM compartment, control animals exhibited more mature T-cells than the PRO 140 animals (40.2% vs. 26.4%, FIG. 6, BM panels E2 quadrants). This occurred while control animals were experiencing severe GvHD while the PRO 140 animals were gaining weight without signs of GvHD. A determination of the absolute number of cells in each quadrant supported this observation (FIG. 6).

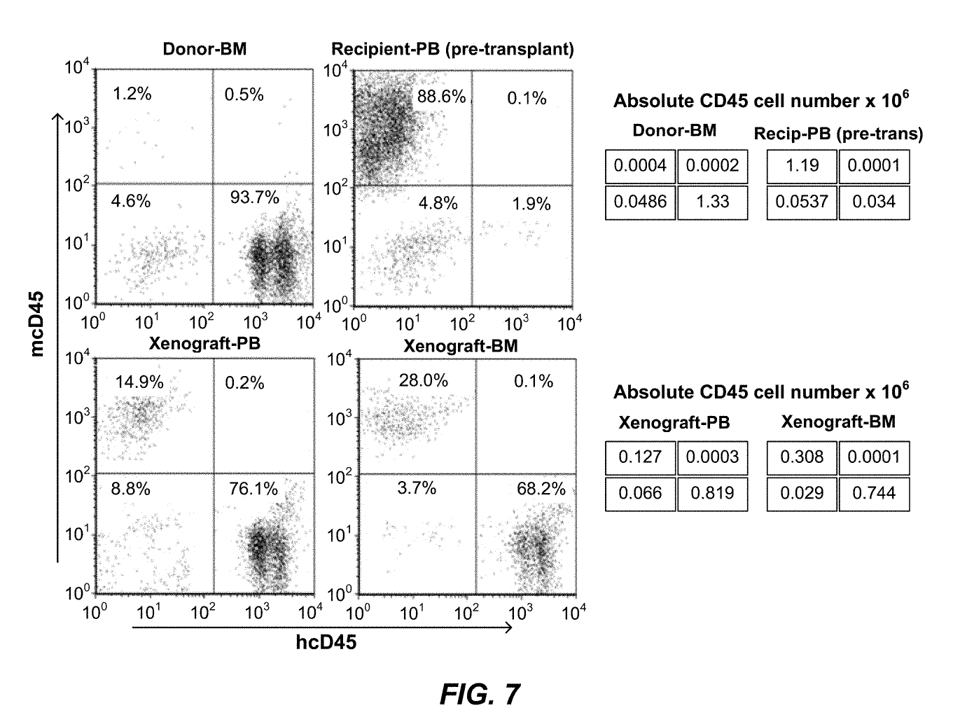

An analysis of engraftment was carried out with PB and BM in the high dose cohort at the time of euthanasia (Day 75) by flow cytometry with antibodies specific for human and mouse CD45. The human donor BM was 93.7% positive for human CD45 and the mouse recipients before engraftment were 88.6% positive for mouse CD45 (FIG. 7, top panels left and right, respectively). Seventy-five days after engraftment, PB in the mice was 76.1% positive for human CD45 and BM was 68.2% positive for human CD45 (FIG. 7, bottom left and right panels, respectively). Mouse hematopoietic cells from PB and BM were 14.9% and 28% respectively. This was consistent with a determination of the absolute number of cells of human or mouse origin (FIG. 7).

It is contemplated that, beyond 70 days, engraftment may continue towards completion, i.e., to achieve mice with humanized immune systems with engraftment levels greater than or equal to about 60%, 65%, 70%, 75%, 80%, 85%, 86%, 87%, 88%, 89%, 90%, 91%, 92%, 93%, 94%, 95%, 96%, 97%, 98%, 99%, or 100% in one or more of mouse bone marrow, mouse spleen, and mouse peripheral blood cells as measured by flow cytometry analysis of engrafted human cells.

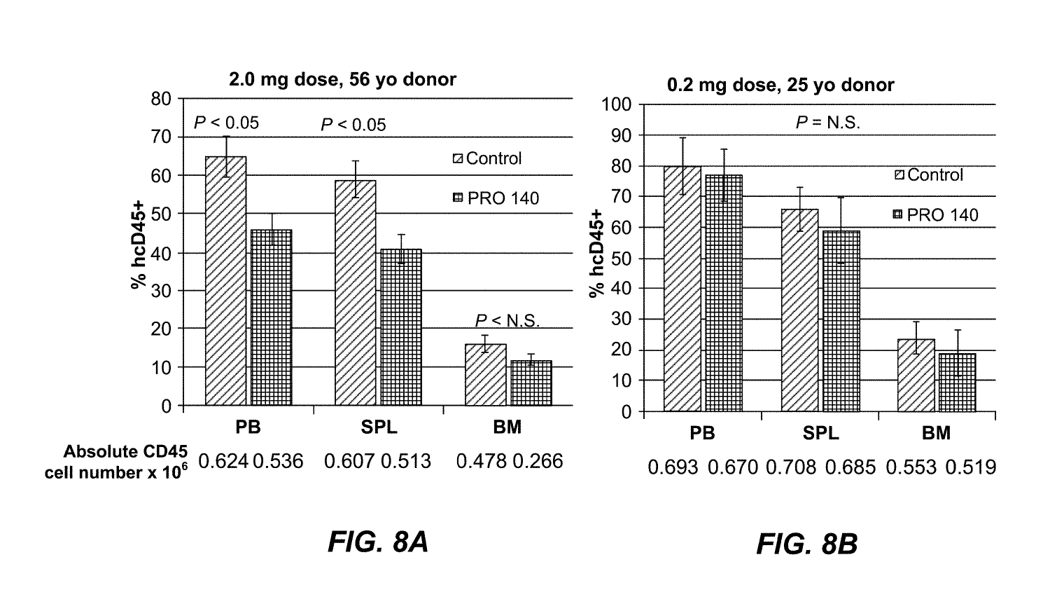

Further analysis of engraftment assessed in peripheral blood and bone marrow at 54 days by flow cytometry with antibodies specific for CD45 (all differentiate hematopoietic cells) and CD3 (mature T Cells) showed more mature T cells in the bone marrow of control animals than of the PRO 140 animals (40.2% vs. 26.4, FIG. 6). The pattern of the flow analysis was also altered in the peripheral blood with the appearance of a new population of cells in the control (GVHD animals, FIG. 6, PRO 140 window E3). It is contemplated that PRO 140 reduced inflammation in the PRO 140 treated animals, leading to lower maturation rates for T cells. At the time of euthanasia, an additional flow analysis was performed with the antibody on peripheral blood, spleen and bone marrow cells (FIG. 8A and FIG. 8B). There was no difference between control and PRO 140 treated animals in the bone marrow, indicating equivalent engraftment. And this indicated that PRO 140 did not inhibit engraftment. Both spleen (58% vs. 41%) and peripheral blood (64% vs. 45%) had significantly more CD45+ cells in control (late stage GVHD) versus Pro 140 treated animals (p<0.05).

This difference was likely attributable to the late stage GvHD ongoing in the control animals at this time. A determination of the absolute number of cells was consistent with these observations (FIG. 8A and FIG. 8B).

### Discussion

The present invention provides for inhibition or blockade of immunomodulatory cell receptors, such as the CCR5 cell receptor, to treat or prevent GVHD. The present invention involves evidence from laboratory animals with substantially or completely reconstituted humanized immune systems that have improved health and longevity relative to laboratory animals that do not receive a CCR5 binding agent. Thus, the invention relates generally to CCR5 binding agents and compositions, and methods of using such agents to treat or prevent GVHD.

Here, the inventor exemplifies the present invention using immunodeficient mice with the targeted IL-2Rynull mutation, namely NSG mice, which have been established as a model of choice for engraftment by HSCT for the study of therapeutic approaches for GVHD. This model allowed evaluation of the role of a potent CCR5 inhibitor, PRO 140, on the role of immune cell trafficking in the pathogenesis of GVHD. Importantly, however, it was found that PRO 140 is not only a robust inhibitor of acute GVHD in this model system, as measured by physical signs, weight loss, and survival curves, but that overall mouse health and longevity was significantly improved. Thus, the inventors also found that treating immunocompromised animals with PRO 140 along with engraftment gives rise to an improved mouse model for studying human immune functionality in healthier, longer-lived mice with substantially, or possibly, completely, reconstituted human immune systems (FIGS. 1 and 2).

The rationale supporting the inventor's GVHD study is based, in part, on the role of CCR5, the G-linked protein receptor for CCL5 (aka RANTES), which is a potent chemokine involved in immune cell trafficking. Immune cell trafficking is believed to be crucial for the development of acute GVHD, which involves cutaneous and organ involvement, including spleen, small intestine and liver, with some involvement of bone marrow and thymus. We did not conduct a histological evaluation of organ involvement in these studies and therefore cannot attribute the effects of PRO 140 to modulation of immune cell trafficking. We plan to do so in follow-on mechanistic studies. Previous murine and human clinical trials have shown that blockade of CCR5 using a small molecule inhibitor, MVR, can reduce the clinical impact of acute GvHD without significantly affecting engraftment.⁸⁻¹⁰ We have previously shown that PRO 140 is a competitive inhibitor of HIV binding to CCR5 without triggering agonist activity, the stimulation of downstream activation markers, or cAMP and tyrosine kinases. These latter characteristics distinguish PRO 140 from MVR.

CCR5 and its natural ligands have also been implicated in transplant organ rejection. Lymphocyte recruitment to tissues involved in GvHD is dependent on CCR5, and migration of CD8+ cells into target organs in murine models is reduced by CCR5 antibody inhibitors resulting in protection against GvHD. CCR5 genetic deletions in mice have resulted in conflicting results in regards to protection from GvHD. In humans, certain CCR5 polymorphisms are protective towards GvHD and correlate with survival in patients with allogeneic bone marrow transplants.

An important question regarding the use of PRO 140 in attempts to abrogate GvHD is whether it would have detrimental effects on engraftment. We found no such effects in PB in early stages of engraftment and in PB and BM in late stages of engraftment. However we did observe significantly more CD45⁺ cells in animals experiencing severe GvHD than in PRO 140 animals at 50 days. There were also more CD45+ cells in PB (64% vs. 46%) and SPL (59% vs. 41%) at the time of euthanasia in the GvHD animals (control group) as compared to PRO 140-treated animals which had no signs of GvHD. There were no differences in CD45+ cells in BM at this time suggesting that PRO 140 did not negatively affect engraftment. There were also more mature T-cells (CD3⁺) in the BM of control (GvHD animals) compared to PRO 140-treated animals at Day 54, when the control animals required sacrifice due to severe GvHD.

We plan to evaluate the observed functional tolerance on the continued dependence of PRO 140 treatment, clonal deletions, and/or the contributions of regulatory cell activity in addition to a potential role for NK cells and other yet to be defined mechanisms. In future studies we will also assess the functional aspects of the human immune cells in the engrafted mice treated with PRO 140. It should be pointed out that here we used a severely immunocompromised mouse model for the production of xeno-GvHD. We plan to evaluate PRO 140 in allogeneic GvHD mouse models in future experiments. This is an important consideration when bone marrow stem cell transplantation is used in patients with blood cell malignancies such as AML. The graft versus cancer (GVL) response often correlates with the GvHD response. In additional experiments in the future we plan to evaluate the GVL response in animals with reduced or eliminated GvHD responses during PRO 140 treatment.

Taken together these data suggest that the CCR5 receptor on engrafted cells is critical for the development of acute GvHD in this model system and that targeting this receptor is a viable approach to mitigating acute GvHD. As this model system has been widely accepted as a reliable model for allogeneic GvHD in humans, we believe that PRO 140 has a place in investigative approaches to resolving acute GvHD in AML and MDS patients undergoing stem cell transplantation. CytoDyn currently is enrolling patients in an FDA approved Phase 2 protocol for this indication.

The present invention answers important questions regarding the use of PRO 140 in attempts to abrogate GVHD, such as whether it would have effects on engraftment. It was found to have no such effects in peripheral blood in early stages of engraftment (FIGS. 5A, 5B, 5C, and 5D) and in peripheral blood and bone marrow in late stages of engraftment (FIG. 7). However it was observed that there were significantly more CD45+ cells in animals experiencing severe GVHD than in PRO 140 animals at 50 days. There were also more CD45+ cells in peripheral blood (64% vs. 46%) and spleen (59% vs. 41%) at the time of euthanasia in the GVHD animals (control group) as compared to PRO 140 treated animals with no signs of GVHD (FIG. 8). There were no differences in CD45+ cells in bone marrow at this time, which suggests that PRO 140 did not negatively affect engraftment (FIG. 8). There were also more mature T cells (CD3+) in the bone marrow of control (GVHD animals) compared to PRO 140 treated animals at Day 54 (FIG. 6), when the control animals required sacrifice (severe GVHD).

Taken together these data suggest that the CCR5 receptor on engrafted cells is critical for the development of acute GVHD in this model system, and that blocking this receptor from recognizing chemokines in the CCR family is a viable approach to, not only mitigating, acute GVHD. Moreover, the significant results achieved by the use of an anti-CCR5 binding agent in engrafted mice in terms of mouse health and longevity, and possibly substantial or complete human hematopoietic engraftment, gives rise to a further improved mouse model for human immune studies. As the NSG model system has been widely accepted as a reliable model for allogeneic GVHD in humans, PRO 140 has a place in investigative approaches to resolving acute GVHD in AML and MDM patients undergoing stem cell transplantation and, possibly, for use in new mouse models for the study of human immune function.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of treating or preventing GVHD comprising administering to a subject in need thereof a competitive inhibitor to a CCR5 cell receptor that does not itself have CCL5 agonist activity.

2. The method according to claim 1, wherein the competitive inhibitor binds to an ECL-2 loop of the CCR5 cell receptor.

3. The method according to claim 1, wherein the competitive inhibitor competes with CCL5 for binding to the CCR5 cell receptor.

4. The method according to claim 1, wherein the competitive inhibitor comprises monoclonal antibody PA14, PRO 140, or CCR5mAb004, or a binding fragment thereof.

5. The method according to claim 1, wherein the competitive inhibitor does not affect cAMP levels when added to CD4+ T cells alone.

6. The method according to claim 1, wherein the competitive inhibitor does not affect chemotaxis of CHO-K1 cells.

7. The method according to claim 1, wherein the subject exhibits at least one of maintained body weight and no physical signs associated with GVHD.

8. (canceled)

9. The method according to claim 7, wherein the subject exhibits no physical signs associated with GVHD within one of thirty days following treatment or seventy days following treatment.

10. (canceled)

11. The method according to claim 1, wherein the GVHD is one of acute GVHD and chronic GVHD.

12. (canceled)

13. The method according to claim 1, wherein the competitive inhibitor is administered to the subject during at least one of before transplant, during transplant, and after transplant.

14. (canceled)

15. (canceled)

16. A method of treating or preventing GVHD comprising administering to a subject in need thereof an anti-CCR5 cell receptor binding agent comprising:
a. a PRO 140 antibody, or binding fragment thereof;
b. a nucleic acid encoding a PRO 140 antibody, or binding fragment thereof;
c. a vector comprising a nucleic acid encoding a PRO 140 antibody, or binding fragment thereof; or
d. a host cell comprising (i) a PRO 140 antibody, or binding fragment thereof, (ii) a nucleic acid encoding a PRO 140 antibody, or binding fragment thereof, or (iii) a vector comprising a nucleic acid encoding a PRO 140 antibody, or binding fragment thereof.

17. The method of claim 16, wherein the PRO 140 antibody, or binding fragment thereof, comprises PRO 140 monoclonal antibody.

18. The method of claim 16, wherein the PRO 140 antibody or binding fragment thereof, comprises a scFv.

19. (canceled)

20. The method according to claim 16, wherein the GVHD is one of acute GVHD and chronic GVHD.

21. (canceled)

22. The method according to claim 16, wherein the anti-CCR5 cell receptor binding agent is administered to the subject before transplant.

23. The method according to claim 16, wherein the anti-CCR5 cell receptor binding agent is administered to the subject during transplant.

24. The method according to claim 16, wherein the anti-CCR5 cell receptor binding agent is administered to the subject after transplant.

25. The method according to claim 16, wherein the anti-CCR5 cell receptor binding agent is administered as a subcutaneous dose.

26. The method according to claim 25, wherein the subcutaneous dose comprises a formulation with the anti-CCR5 cell receptor binding agent provided at a concentration of about 175 mg/mL.

27. A therapeutic composition for treatment or prevention of GVHD comprising an anti-CCR5 cell receptor binding agent to a CCR5 cell receptor that does not affect cAMP levels when added to CD4+ T cells alone or does not affect chemotaxis of CHO-K1 cells.
